(19) United States
(12) Patent Application Publication
(10) Pub. No.: US 2020/0179450 A1
(43) Pub. Date: Jun. 11, 2020

(54) T-CELL ANTIGEN TARGETED CHIMERIC ANTIGEN RECEPTOR (CAR) AND USES IN CELL THERAPIES

(71) Applicants: EMORY UNIVERSITY, Atlanta, GA (US); CHILDREN'S HEALTHCARE OF ATLANTA, INC., Atlanta, GA (US)

(72) Inventors: H. Trent SPENCER, Atlanta, GA (US); Christopher DOERING, Atlanta, GA (US); Sunil RAIKAR, Atlanta, GA (US); Lauren FLEISCHER, Atlanta, GA (US)

(21) Appl. No.: 16/622,071

(22) PCT Filed: Jun. 12, 2018
(86) PCT No.: PCT/US2018/037160

§ 371 Date: Dec. 12, 2019

Related U.S. Application Data
(60) Provisional application No. 62/518,588, filed on Jun. 12, 2017.

(51) Int. Cl. A61K 35/17; C07K 14/725; C07K 16/28; C12N 15/86; C07K 14/715

(52) U.S. Cl. CPC A61K 35/17; C07K 14/7051; C12N 2740/16043; C12N 15/86; C07K 14/7155; C07K 16/28

(57) ABSTRACT

This disclosure relates to engineered cells, such as T-cells, comprising targeted chimeric antigen receptors. In certain embodiments, T-cell targeted chimeric antigen receptors (CAR) are expressed at higher levels when endogenous expression of a T-cell antigen is knocked-down or reduced in the T-cells. In certain embodiments, the engineered cells are immunoregulatory cells genetically modified to prevent or reduce T-cell antigen expression, or the immunoregulatory cells contain a nucleic acid that reduces or knocksdown T-cell mRNA expression, under conditions such that reduced expression of the T-cell antigen results in an increased expression of a chimeric antigen receptor compared to similarly situated immunoregulatory cells wherein the expression of the T-cell antigen is not altered or reduced. In certain embodiments, T-cell antigens include, but are not limited to, CD5, CD7 and CD3.

### CROSS-REFERENCE TO RELATED APPLICATIONS

This application claims the benefit of U.S. Provisional Application No. 62/518,588 filed Jun. 12, 2017. The entirety of this application is hereby incorporated by reference for all purposes.

### STATEMENT REGARDING FEDERALLY SPONSORED RESEARCH OR DEVELOPMENT

This invention was made with government support under grant 1R43CA192710-01 awarded by the National Institutes of Health. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy, and is hereby incorporated by reference into the specification. The name of the text file containing the Sequence Listing is 17172PCT_ST25.txt. The text file is 11 KB, was created on Jun. 12, 2018, and is being submitted electronically via EFS-Web.

### BACKGROUND

Adoptive transfer of genetically modified T cells is a promising approach for generating antitumor immune responses. Administering a preparative chemotherapy regimen followed by autologous T cells genetically engineered to express a chimeric antigen receptor (CAR) that recognized the B-cell antigen CD19 is reported to regress lymphoma. Kochenderfer et al., Blood. 2010, 116(20):4099-102. However, the treatment of T-cell malignancies is complicated by the lack of a T-lymphoblast specific surface antigen. As a result, CAR T cells generated to target malignant T cells are at risk of fratricide, i.e., CAR T cell self-destruction. Therefore, their activation against targeted cancer T cells is compromised. Thus, there is a need to identify improved methods.

CD5 is a pan T-cell marker that is commonly overexpressed in most T-cell malignancies. CD5 expression by normal cells is believed to be restricted to thymocytes, peripheral T cells, and a minor subpopulation of B lymphocytes, called B-1 cells. Chen et al. report preclinical targeting of aggressive T-cell malignancies using anti-CD5 chimeric antigen receptor. Leukemia, 2017, 31(10):2151-2160. This report indicates fratricide among the engineered CAR T cells due to inherent CD5 expression. See also Mamonkin et al., Blood. 2015, 126(8):983-92, WO 2016/172606, WO 2016/138491, WO 2017/146767, and ClinicalTrials.gov Identifier NCT03081910 entitled Autologous T-Cells Expressing a Second Generation CAR for Treatment of T-Cell Malignancies Expressing CD5 Antigen (MAGENTA).

References cited herein are not an admission of prior art.

### SUMMARY

This disclosure relates to engineered cells, such as T-cells, comprising targeted chimeric antigen receptors. In certain embodiments, T-cell targeted chimeric antigen receptors (CAR) are expressed at higher levels when endogenous expression of a T-cell antigen is knocked-down or reduced in the T-cells. In certain embodiments, the engineered cells are immunoregulatory cells genetically modified to prevent or reduce T-cell antigen expression, or the immunoregulatory cells contain a nucleic acid that reduces or knocks-down T-cell mRNA expression, under conditions such that reduced expression of the T-cell antigen results in an increased expression of a chimeric antigen receptor compared to similarly situated immunoregulatory cells wherein the expression of the T-cell antigen is not altered or reduced. In certain embodiments, T-cell antigens include, but are not limited to, CD5, CD7 and CD3.

In certain embodiments, disclosure relates to engineered cells comprising T-cells antigen targeted chimeric antigen receptors. In certain embodiments, the engineered cells are immunoregulatory cells genetically modified to prevent or reduce T-cells antigen expression or the immunoregulatory cells contain a nucleic acid that reduces or knocks-down T-cells antigen mRNA expression. In certain embodiments, the disclosure relates to methods of managing conditions associated with abnormal T cell conditions, such as treating a T cell malignancy comprising administering engineered cells with T-cells antigen targeted chimeric antigen receptors (CARS), reducing natural T-cells antigen surface expression, to a subject diagnosed with a T cell malignancy. In certain embodiments, reduced expression of T-cells antigen results in an increased expression of a chimeric antigen receptor comprising a T-cells antigen recognition domain on the immunoregulatory cells, such as T-cells, compared to similarly situated immunoregulatory cells wherein the expression of T-cells antigen is not altered or reduced.

In certain embodiments, disclosure relates to engineered cells comprising CD5, CD7 and/or CD3 targeted chimeric antigen receptors. In certain embodiments, the engineered cells are immunoregulatory cells genetically modified to prevent or reduce CD5, CD7 and/or CD3 expression or the immunoregulatory cells contain a nucleic acid that reduces or knocks-down CD5, CD7 and/or CD3 mRNA expression. In certain embodiments, the disclosure relates to methods of managing conditions associated with abnormal T cell conditions, such as treating a T cell malignancy comprising administering engineered cells with CD5, CD7 and/or CD3 targeted chimeric antigen receptors (CARS), reducing natural CD5, CD7 and/or CD3 surface expression, to a subject diagnosed with a T cell malignancy. In certain embodiments, reduced expression of CD5, CD7 and/or CD3 results in an increased expression of a chimeric antigen receptor comprising a CD5, CD7 and/or CD3 antigen recognition domain on the immunoregulatory cells, such as T-cells, compared to similarly situated immunoregulatory cells wherein the expression of CD5, CD7 and/or CD3 is not altered or reduced.

In certain embodiments, the present disclosure provides CD5, CD7 and/or CD3 targeted chimeric antigen receptors (CARS) for hematologic malignancies, compositions and methods of use thereof. In certain embodiment, the disclosure provides an engineered chimeric antigen receptor polypeptide, the polypeptide comprising: a signal peptide, a CD5, CD7 and/or CD3 antigen recognition domain, a hinge region, a transmembrane domain, at least one co-stimulatory domain, and a signaling domain.

In certain embodiments, the disclosure provides an engineered chimeric antigen receptor polypeptide or polynucleotide that encodes for a chimeric antigen receptor polypeptide having an antigen recognition domain selective for CD5, such as a CD5 targeted scFv. In certain embodiments, the CD5 targeted scFv has SEQ ID NO: 8, or variants thereof. In certain embodiments, variants have greater than 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70% or more identity to SEQ ID NO: 8. In certain embodiments variants are 1 or 2 mutations, deletions, or insertions outside CDR1, CDR2, or CDR3 of the light or heavy chain variable region. In certain embodiments variants are 3 or 4 mutations, deletions, or insertions outside CDR1, CDR2, or CDR3 of the light or heavy chain variable region. In certain embodiments variants are 1 or 2 mutations, deletions, or insertions inside CDR1, CDR2, or CDR3 of the light or heavy chain variable region.

In another embodiment, the disclosure provides an engineered cell expressing any of the chimeric antigen receptor polynucleotides or polypeptides described above. In certain embodiments, the engineered cell is an immunoregulatory cell, such as a T-cell or NK cell. In certain embodiments, the T-cells are compositions of purified gamma delta T cells, alpha beta T cells, or combinations thereof.

In certain embodiments, this disclosure relates to treating cancer comprising: isolating immunoregulatory cells, such as T-cells, from a subject; modifying the isolated immunoregulatory cells, such as T-cells, such that expression of a T-cell antigen is reduced; inserting a vector or DNA into the immunoregulatory cells, such as T-cells, wherein the vector of DNA encodes and expresses a chimeric antigen receptor comprising a T-cell antigen recognition domain under conditions such that the immunoregulatory cells, such as T-cells, express the T-cell antigen recognition domain providing transduced or engineered cells, such as transduced or engineered T-cells; and administering an effective amount of transduced or engineered cells, to the subject, optionally in combination with IL-2, to the subject.

In certain embodiments, this disclosure relates to treating cancer comprising: isolating immunoregulatory cells, such as T-cells, from a subject; modifying the isolated immunoregulatory cells, such as T-cells, such that expression of CD5, CD7 and/or CD3 is reduced; inserting a vector or DNA into the immunoregulatory cells, such as T-cells, wherein the vector or DNA encodes and expresses a chimeric antigen receptor comprising a CD5, CD7 and/or CD3 antigen recognition domain under conditions such that the immunoregulatory cells, such as T-cells, express the CD5, CD7 and/or CD3 antigen recognition domain providing transduced or engineered cells, such as transduced or engineered T-cells; and administering an effective amount of transduced or engineered cells, to the subject, optionally in combination with IL-2, to the subject.

In certain embodiments, reduced expression of T-cell antigen results in an increased expression of a chimeric antigen receptor comprising a T-cell antigen recognition domain on the immunoregulatory cells, such as T-cells, compared to similarly situated immunoregulatory cells wherein the expression of T-cell antigen is not altered or reduced.

In certain embodiments, reduced expression of CD5, CD7 and/or CD3 results in an increased expression of a chimeric antigen receptor comprising a CD5, CD7 and/or CD3 antigen recognition domain on the immunoregulatory cells, such as T-cells, compared to similarly situated immunoregulatory cells wherein the expression of CD5, CD7 and/or CD3 is not altered or reduced.

In certain embodiments, modifying the isolated immunoregulatory cells, such as T-cells, such that expression of CD5, CD7 and/or CD3 is reduced comprises inserting a vector or DNA into the immunoregulatory cells, such as T-cells, wherein the vector or DNA encodes and expresses a Cas nuclease, e.g. Cas9, and a guide RNA that targets a sequence for cleaving, nicking, or blocking expression of the CD5, CD7 and/or CD3 gene or mRNA. In certain embodiments, the guide RNA comprises AGCGGTTGCAGAGACCCCAT (SEQ ID NO: 5) for targeting CD5.

In certain embodiments, modifying the isolated immunoregulatory cells, such as T-cells, such that expression of CD5, CD7 and/or CD3 is reduced comprises inserting into the immunoregulatory cells, such as T-cells, mRNA that encodes a Cas nuclease and a guide RNA that targets a sequence for cleaving, nicking, or blocking expression of the CD5, CD7 and/or CD3 gene or mRNA. In certain embodiments, guide RNA comprises AGCGGTTGCAGAGACCCCAT (SEQ ID NO: 5) for targeting CD5.

In certain embodiments, modifying the immunoregulatory cells, such as T-cells, such that expression of CD5, CD7 and/or CD3 is reduced comprises inserting a vector or mRNA into the immunoregulatory cells, such as T-cells, wherein the vector or mRNA encodes and expresses a double stranded or short hairpin RNA capable of reducing CD5, CD7 and/or CD3 mRNA expression. In certain embodiments, modifying the isolated immunoregulatory cells, such as T-cells, such that expression of CD5, CD7 and/or CD3 is reduced comprises inserting double stranded RNA oligonucleotides into the T-cells, e.g., into the cytosol, wherein the RNA is capable of reducing CD5, CD7 and/or CD3 mRNA expression by RNA interference (RNAi). In certain embodiments, engineered immunoregulatory cells, such as T-cells, are considered to be expression reduced CD5 modified cells as described above.

In certain embodiments, the T-cells are obtained from autologous peripheral blood lymphocytes (PBL) of the subject, e.g., isolated by leukapheresis.

In certain embodiments, administering an effective amount of transduced immunoregulatory cells, such as T-cells, to the subject is after administering a lymphodepleting regimen to the subject. In certain embodiments, the lymphodepleting regimen is non-myeloablative or myeloablative. In certain embodiments, the lymphodepleting regimen comprises administering cyclophosphamide, fludarabine, or a combination thereof.

In certain embodiments, this disclosure relates to immunoregulatory cells, such as T-cells, comprising CD5, CD7 and/or CD3 targeted chimeric antigen receptors and uses for targeting T-cell malignancies by using CD5, CD7 and/or CD3 CRISPR-edited immunoregulatory cell line, such as T-cell lines. In certain embodiments, this disclosure relates to immunoregulatory cells, such as T-cells, comprising 1) an engineered chimeric antigen receptor polypeptide, the polypeptide comprising: a CD5, CD7 and/or CD3 antigen recognition domain, a hinge region, a transmembrane domain, at least one co-stimulatory domain, and a signaling domain, and 2) CD5, CD7 and/or CD3 gene which is deleted or mutated such that CD5, CD7 and/or CD3 surface expression is reduced or eliminated. In certain embodiments, said CD5, CD7 and/or CD3 antigen recognition domain comprises the binding portion or variable region of a monoclonal antibody that specifically binds CD5, CD7 and/or CD3, such as a CD5, CD7 and/or CD3 targeted scFv-CAR.

In certain embodiments, this disclosure relates to knocking-out surface expression of the target antigen in CAR T cells using CRISPR-Cas9 genome editing. In certain embodiments, the disclosure contemplates CD5, CD7 and/or CD3 -CRISPR-edited T cells with decreased self-activation when expressing a CD5, CD7 and/or CD3 -CAR compared to that of CD5, CD7 and/or CD3-positive T cells.

In certain embodiments, this disclosure relates to reduced expression of CD5 in immunoregulatory cells, such as T-cells, resulting in an increased expression of a chimeric antigen receptor comprising a CD5, CD7 and/or CD3 antigen recognition domain on the immunoregulatory cells, such as T-cells, compared to immunoregulatory cells, such as T-cells, wherein the expression of CD5, CD7 and/or CD3 is not altered or reduced.

In certain embodiments, the CD5 antigen recognition domain is the homo sapiens T-cell surface glycoprotein CD5 isoform 1, e.g., comprises a polypeptide that is selective for SEQ ID NO: 1,

RLSWYDPDFQARLTRSNSKCQGQLEVYLKDGWHMVCSQSWGRSSKQWEDP
SQASKVCQRLNCGVPLSLGPFLVTYTPQSSIICYGQLGSFSNCSHSRNDM
CHSLGLTCLEPQKTTPPTTRPPPTTTPEPTAPPRLQLVAQSGGQHCAGVV
EFYSGSLGGTISYEAQDKTQDLENFLCNNLQCGSFLKHLPETEAGRAQDP
GEPREHQPLPIQWKIQNSSCTSLEHCFRKIKPQKSGRVLALLCSGFQPKV
QSRLVGGSSICEGTVEVRQGAQWAALCDSSSARSSLRWEEVCREQQCGSV
NSYRVLDAGDPTSRGLFCPHQKLSQCHELWERNSYCKKVFVTCQDPNP

In certain embodiments, the disclosure provides for a method of producing an engineered cell expressing a chimeric antigen receptor polypeptide or polynucleotide having an antigen recognition domain that specifically binds CD5, CD7 and/or CD3 and a CD5, CD7 and/or CD3 gene comprising a mutation, addition, or deletion such that CD5, CD7 and/or CD3 is not expressed on the engineered cells, or provided expression reduced CD5, CD7 and/or CD3 modified cells. In certain embodiments, the method includes (i) providing peripheral blood cells or cord blood cells; (ii) introducing the aforementioned polynucleotide into the aforementioned cells; (iii) expanding the cells of step (ii); and isolating the cells of step (iii) to provide said engineered cells or expression reduced CD5, CD7 and/or CD3 modified cells. In certain embodiments, the method includes (i) providing peripheral blood cells or cord blood cells; (ii) introducing the aforementioned polypeptide or polynucleotide encoding CD5, CD7 and/or CD3 targeted chimeric antigen receptor and optionally a Cas nuclease, e.g. Cas9, and gRNA that targets the CD5, CD7 and/or CD3 gene or mRNA into the aforementioned cells; (iii) expanding the cells of step (ii); and isolating the cells of step (iii) to provide said engineered cells or expression reduced CD5, CD7 and/or CD3 modified cells.

In certain embodiments, the disclosure provides a method of producing an engineered cell, or expression reduced CD5, CD7 and/or CD3 modified cells, expressing a chimeric antigen polypeptide or polynucleotide having an antigen recognition domain selective for CD5, CD7 and/or CD3. In certain embodiments, the method includes (i) providing placental cells, embryonic stem cells, induced pluripotent stem cells, or hematopoietic stem cells; (ii) introducing the aforementioned polynucleotide, e.g., that encodes a CD5, CD7 and/or CD3 targeted scFv-CAR, into the cells of step (i); (iii) expanding the cells of step (ii); and (iv) isolating the cells of step (iii) to provide said engineered cells, or expression reduced CD5, CD7 and/or CD3 modified cells.

In certain embodiments, the disclosure provides a method of reducing the number of immunoregulatory cells having CD5, CD7 and/or CD3 expressed on the surface of the cells. The method includes (i) contacting said immunoregulatory cells with an effective amount of an engineered cell, or expression reduced CD5, CD7 and/or CD3 modified cells, expressing a CAR polypeptide having a CD5, CD7 and/or CD3 antigen recognition domain; and (ii) optionally, assaying for the reduction in the number of immunoregulatory cells.

In one embodiment, the disclosure provides a method of treating a cell proliferative disease. The method includes administering to a patient in need thereof a therapeutically effective amount of an engineered cell, or expression reduced CD5, CD7 and/or CD3 modified cells, expressing a CAR polypeptide having a T-cell targeted antigen recognition domain, e.g., that encodes a CD5, CD7 and/or CD3 scFv-CAR and optionally contains a Cas nuclease and gRNA that targets the CD5, CD7 and/or CD3 gene or mRNA expression.

In certain embodiments, the disclosure provides a method of treating an autoimmune disease. The method includes (i) administering to a patient in need thereof a therapeutically effective amount of an engineered cell, or expression reduced CD5, CD7 and/or CD3 modified cells, expressing a CAR polypeptide having a CD5, CD7 and/or CD3 targeted antigen recognition domain.

In certain embodiments, the disclosure provides engineered cells, or expression reduced CD5, CD7 and/or CD3 modified cells, expressing a CAR polypeptide having a CD5, CD7 and/or CD3 antigen recognition domain for use in the treatment of a cell proliferative disease. The use includes administering said engineered cells, or expression reduced CD5, CD7 and/or CD3 modified cells, or combinations thereof to a patient in need thereof.

In some embodiments, CARs typically include at least one of intracellular signaling, hinge and/or transmembrane domains. First-generation CARs include CD3zeta as an intracellular signaling domain, whereas second-generation CARs include a single co-stimulatory domain derived from, for example, without limitation, CD28 or 4-IBB. Third generation CARs include two co-stimulatory domains, such as, without limitation, CD28, 4-1BB (also known CD137) and OX-40, and any other co-stimulatory molecules.

In some embodiments, a polynucleotide encoding a CAR having a CD5, CD7 and/or CD3 antigen recognition domain is part of a gene in an expression cassette. In a preferred embodiment, the expressing gene or the cassette may include an accessory gene, gene encoding a fluorescent protein, or a tag or a part thereof. The accessory gene may be an inducible suicide gene or a part thereof, including, but not limited to, caspase 9 gene. The “suicide gene” ablation approach improves safety of the gene therapy and kills cells only when activated by a specific compound or a molecule. In some embodiments, the epitope tag is a c-myc tag, streptavidin-binding peptide (SBP), truncated EGFR gene (EGFRt) or a part or a combination thereof.

### BRIEF DESCRIPTION OF THE DRAWINGS

FIG. 1A illustrates CAR structures containing the CD5-directed variable lymphocyte receptor (VLR) or single chain variable fragment (scFv). CAR structures with CD28 containing a scFv (left) or VLR (right) as the antigen recognition domain are shown.

FIG. 1B illustrates the bicistronic transgene sequences used for expressing enhanced green fluorescent protein (eGFP) and the CD5-CARs using a P2A self-cleaving sequence. It includes a 5′ long terminal repeat (LTR), human ubiquitin C promoter (hUBC), eGFP sequence, P2A sequence, an interleukin-2 signal peptide (IL-2 SP), the CD5-VLR (top) or CD5-scFv (bottom), a myc epitope tag, the CD28 region, the CD3zeta intracellular domain and a 3′ LTR.

FIG. 2A shows western blot using anti-CD3zeta antibody on whole cell lysates of NK-92 cells shows the presence of CD5-VLR-CAR and CD5-scFv-CAR protein in the sorted and expanded cells. NK-92 cells were transduced with the eGFP-P2 A-CD5-scFv-CAR lentiviral vector and sorted for GFP expressing cells. After two rounds of sorting, an enriched population of CAR-expressing NK-92 cells was generated with 99% eGFP expression.

FIG. 2B shows data where both CD5-CAR expressing NK-92 cells were mixed with CD5-positive target cells Jurkat at various Effector: Target ratios and the percent cytotoxicity was measured by flow cytometry.

FIG. 2C shows data for MOLT-4. CD5-CAR modified NK-92 cells showed a significantly greater cytotoxicity (p<0.01) against the CD5-positive Jurkat and MOLT-4 cells when compared to unmodified NK-92 cells in a 4 hour assay. This data indicates NK-92 cell mediated cytotoxicity against a CD5-positive T-ALL cell line using CD5-CARs.

FIG. 2D shows data indicating no increase in cytotoxicity is seen when CD5-CAR NK-92 cells are cultured with CD5-negative 697 cells.

FIG. 3A illustrates a method where Jurkat T cells were transduced with lentiviral vectors encoding either a scFv- or VLR-based CD5-CAR with co-expression of eGFP. The Jurkat T cell activation assay shows time points for measurement of T-cell activation and Western blot analysis.

FIG. 3B shows data on activation measured by surface CD69 expression four days after transduction increased as the amount of viral vector increased. Greater activation was observed in the CD5-VLR-CAR Jurkat group.

FIG. 3C shows data on the percentage of activated cells was compared to the vector copy number (VCN) obtained for each transduced population of cells. The inset to the figure defines each group.

FIG. 3D shows data on CD69 expression was measured 4 and 12 d after transduction, which showed activation decreased over time in both CD5-CAR expressing Jurkat T cell groups.

FIG. 4A shows data on CD5 knockout in Jurkat T cells using CRISPR-Cas9 genome editing. CD5 expression, measure by flow cytometry, in Jurkat T cells five days following mock transfection or transfection with plasmid encoding Cas9 and one of three different gRNA target sequences. Histogram plots for CD5 expression in mock transfected and transfected Jurkat T cells are shown along a single axis.

FIG. 4B shows an overlay image of histogram plots of CD5 expression in Naive Jurkat T cells and flow-sorted CD5-negative Jurkat T cells that were transfected with the CD5-CRISPR gRNA #2.

FIG. 4C shows representative sequencing traces from Naive (top left) CCTGCTGGGGATGCTGGGTGAGT (SEQ ID NO: 2) and sorted CD5-edited (top right) CCGGTGGGGGGTGGGGGGGGA (SEQ ID NO: 3) Jurkat T cell genomic DNA PCR amplified for CD5, sequence the gene from genomic DNA.

FIG. 4D shows a TIDE analysis of the frequency of indels within the CD5 gene after the predicted break-site generated by Cas9. Results show 77% CD5-negative cells were edited with 27% having a—1 deletion.

FIG. 5A shows percentage of eGFP positive cells. CD5-edited CD5-CAR-modified Jurkat T cells have reduced self-activation and increased CD5-CAR expression. Naive (white) and CD5-edited Jurkat T cells (black) were transduced with eGFP-P2 A-CD5-VLR-CAR, eGFP-P2 A-CD5-scFv-CAR or control eGFP-P2 A-BCL-VLR-CAR lentiviral vectors at MOIs 1, 10 and 20. Polybrene was not used during transduction, which provided a greater separation in transduction efficiency between MOIs of 1 and 10. Transduction efficiency, measured by eGFP-positive cells, of each CAR vector at MOIs 1, 10 and 20 in both populations of Jurkat T cells.

FIG. 5B shows data on CD5 expression in both populations of Jurkat T cells transduced with each CAR vector at each MOI.

FIG. 5C shows data on the activation was measured by monitoring CD69 expression and transduction efficiency measured by eGFP expression. A correlation exists between activation and eGFP expression in CD5-CAR-transduced Jurkat T cells. Non-edited CD5-CAR-modified cells have increased T-cell activation compared to CD5-edited CD5-CAR-modified cells.

FIG. 5D shows western blots on whole cell lysates showing CD3zeta expression in non-edited Jurkat T cells (left) and CD5-edited Jurkat T cells (right) when transduced with the VLR-CAR vector. Endogenous CD3zeta is represented by the 18 kDa bands and CD3zeta in the CAR construct is represented by the 48, kDa band in the CD5-VLR-CAR construct. eGFP, CD5 and CD69 surface expression were measured by flow cytometry.

FIG. 6A shows data indicating CD5-edited CD5-CAR-modified effector cells in culture with naive target T cells stimulates effector cell activation and target cell down-regulation of CD5. Naïve and CD5-edited Jurkat T cells were transduced with eGFP-P2A-CD5-scFv-CAR or eGFP-P2A-CD5-VLR-CAR lentiviral vectors at MOI 5. Polybrene was not used during transduction. Target naive Jurkat T cells were labeled with VPD450. On day five post-transduction, effector cells were cultured with labeled target cells at E:T ratios 2:1, 1:1 and 1:5. The cells were analyzed by flow cytometry 24 hours later. White bars signify non-edited effector cells; black bars signify CD5-edited effector cells. Experiments were performed with three replicates and error bars represent standard deviation from the mean. Percent of baseline CD5 expression in target Jurkat T cells cultured with non-edited and CD5-edited effector Jurkat T cells expressing the CD5-scFv-CAR. CD5 expression in target cells cultured alone (gray bar) was used as baseline and set at 100%.

FIG. 6B shows data for the CD5-VLR-CAR.

FIG. 6C shows data on T-cell activation of non-edited and CD5-edited effector Jurkat T cells expressing the CD5-scFv-CAR when cultured alone and in culture with target Jurkat T cells.

FIG. 6D shows data on CD5-VLR-CAR.

FIG. 7A shows data on non-edited Jurkat T cells with CD5-scFv-CAR MOI 5.

FIG. 7B shows data on CD5-edited Jurkat T cells with CD5-scFv-CAR MOI 5.

FIG. 7C shows data indicating antigen editing results in an increase in CAR expression.

FIG. 8A shows Western blots of non-edited Jurkat T cells whole cell lysates.

FIG. 8B shows Western blots of CD5-edited Jurkat T cells whole cell lysates.

FIG. 8C show data on Western blot quantification indicating increased CAR expression in CD5-edited T cells.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

Prior to describing the various embodiments, the following definitions are provided and should be used unless otherwise indicated. Further, headings provided herein are for convenience only and do not interpret the scope or meaning of the claims.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

Unless the context requires otherwise, throughout the specification and claims which follow, the word “comprise” and variations thereof, such as, “comprises,” “comprising” “including,” “containing,” or “characterized by,” are to be construed in an open, inclusive sense, that is, as “including, but not limited to” and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “comprising” in reference to a peptide having an amino acid sequence refers a peptide that may contain additional N-terminal (amine end) or C-terminal (carboxylic acid end) amino acids, i.e., the term is intended to include the amino acid sequence within a larger peptide. The term “consisting of” in reference to a peptide having an amino acid sequence refers a peptide having the exact number of amino acids in the sequence and not more or having not more than a range of amino acids expressly specified in the claim. In certain embodiments, the disclosure contemplates that the “N-terminus of a peptide may consist of an amino acid sequence,” which refers to the N-terminus of the peptide having the exact number of amino acids in the sequence and not more or having not more than a range of amino acids specified in the claim however the C-terminus may be connected to additional amino acids, e.g., as part of a larger peptide. Similarly, the disclosure contemplates that the “C-terminus of a peptide may consist of an amino acid sequence,” which refers to the C-terminus of the peptide having the exact number of amino acids in the sequence and not more or having not more than a range of amino acids specified in the claim however the N-terminus may be connected to additional amino acids, e.g., as part of a larger peptide.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. For example, the polypeptides GGGGGG and GGGGT have a sequence identity of 4 out of 5 or 80%. For example, the polypeptides GGGPPP and GGGAPPP have a sequence identity of 6 out of 7 or 85%. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar—S T N Q. The amino acid groups are also considered conserved substitutions.

### Chimeric Antigen Receptor Polypeptides

In certain embodiments, the disclosure provides a chimeric antigen receptor (CAR) polypeptide having a signal peptide, a T cell antigen recognition domain, e.g., CD5, CD7, and/or CD3 antigen recognition domain, a hinge region, a transmembrane domain, at least one co-stimulatory domain, and a signaling domain.

As used herein, the terms “peptide,” “polypeptide,” and “protein” are used interchangeably, and refer to a compound having amino acid residues covalently linked by peptide bonds. A protein or peptide must contain at least two amino acids, and no limitation is placed on the maximum number of amino acids. Polypeptides include any peptide or protein having two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types.

“Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof

A “signal peptide” includes a peptide sequence that directs the transport and localization of the peptide and any attached polypeptide within a cell, e.g. to a certain cell organelle (such as the endoplasmic reticulum) and/or the cell surface. The signal peptide is a peptide of any secreted or transmembrane protein that directs the transport of the polypeptide of the disclosure to the cell membrane and cell surface, and provides correct localization of the polypeptide of the present disclosure. In particular, the signal peptide of the present disclosure directs the polypeptide of the present disclosure to the cellular membrane, wherein the extracellular portion of the polypeptide is displayed on the cell surface, the transmembrane portion spans the plasma membrane, and the active domain is in the cytoplasmic portion, or interior of the cell. In one embodiment, the signal peptide is cleaved after passage through the endoplasmic reticulum (ER), i.e. is a cleavable signal peptide. In an embodiment, the signal peptide is human protein of type I, II, III, or IV. In an embodiment, the signal peptide includes an immunoglobulin heavy chain signal peptide.

The “antigen recognition domain” includes a polypeptide that is selective for an antigen, receptor, peptide ligand, or protein ligand of the target; or a polypeptide of the target. In one embodiment, the antigen recognition domain includes the binding portion or variable region of a monoclonal or polyclonal antibody directed against (selective for) the target. In one embodiment, the antigen recognition domain includes fragment antigen-binding fragment (Fab). In another embodiment, the antigen recognition domain includes a single-chain variable fragment (scFV). scFV is a fusion protein of the variable regions of the heavy (VH) and light chains (VL) of immunoglobulins, connected with a short linker peptide. In another embodiment, the antigen recognition domain includes ligands that engage their cognate receptor. In another embodiment, the antigen recognition domain is humanized. It is understood that the antigen recognition domain may include some variability within its sequence and still be selective for the targets disclosed herein. Therefore, it is contemplated that the polypeptide of the antigen recognition domain may be at least 95%, at least 90%, at least 80%, or at least 70% identical to the antigen recognition domain polypeptide disclosed herein and still be selective for the targets described herein and be within the scope of the disclosure.

The hinge region is a sequence positioned between for example, including, but not limited to, the chimeric antigen receptor, and at least one co-stimulatory domain and a signaling domain. The hinge sequence may be obtained including, for example, from any suitable sequence from any genus, including human or a part thereof. Such hinge regions are known in the art. In one embodiment, the hinge region includes the hinge region of a human protein including CD-8 alpha, CD28, 4- IBB, OX40, CD3-zeta, T cell receptor a or β chain, a CD3 zeta chain, CD28, CD3s, CD45, CD4, CD5, CD8, CD9, CD16, CD22, CD33, CD37, CD64, CD80, CD86, CD134, CD137, ICOS, CD154, functional derivatives thereof, and combinations thereof. In one embodiment, the hinge region includes the CD8 a hinge region. In some embodiments, the hinge region includes one selected from, but is not limited to, immunoglobulin (e.g. IgG1, IgG2, IgG3, IgG4, and IgD).

The transmembrane domain includes a hydrophobic polypeptide that spans the cellular membrane. In particular, the transmembrane domain spans from one side of a cell membrane (extracellular) through to the other side of the cell membrane (intracellular or cytoplasmic). The transmembrane domain may be in the form of an alpha helix or a beta barrel, or combinations thereof. The transmembrane domain may include a polytopic protein, which has many transmembrane segments, each alpha-helical, beta sheets, or combinations thereof. In one embodiment, the transmembrane domain that naturally is associated with one of the domains in the CAR is used. In another embodiment, the transmembrane domain can be selected or modified by amino acid substitution to avoid binding of such domains to the transmembrane domains of the same or different surface membrane proteins to minimize interactions with other members of the receptor complex. For example, a transmembrane domain includes a transmembrane domain of a T-cell receptor a or β chain, a CD3 zeta chain, CD28, CD3s, CD45, CD4, CD5, CD8, CD9, CD16, CD22, CD33, CD37, CD64, CD80, CD86, CD134, CD137, ICOS, CD154, functional derivatives thereof, and combinations thereof. The artificially designed transmembrane domain is a polypeptide mainly comprising hydrophobic residues such as leucine and valine. In one embodiment, a triplet of phenylalanine, tryptophan and valine is found at each end of the synthetic transmembrane domain. In one embodiment, the transmembrane domain is the CD8 transmembrane domain. In another embodiment, the transmembrane domain is the CD28 transmembrane domain. Such transmembrane domains are known in the art.

The signaling domain and co-stimulatory domain include polypeptides that provide activation of an immune cell to stimulate or activate at least some aspect of the immune cell-signaling pathway. In an embodiment, the signaling domain includes the polypeptide of a functional signaling domain of CD3 zeta, common FcR gamma (FCER1G), Fc gamma RIIIA, FcR beta (Fc Epsilon Rib), CD3 gamma, CD3 delta, CD3 epsilon, CD79a, CD79b, DNAX-activating protein 10 (DAP10), DNAX-activating protein 12 (DAP12), active fragments thereof, functional derivatives thereof, and combinations thereof. Such signaling domains are known in the art. In an embodiment, the CAR polypeptide further includes one or more co-stimulatory domains. In an embodiment, the co-stimulatory domain is a functional signaling domain from a protein including OX40, CD27, CD28, CD30, CD40, PD-1, CD2, CD7, CD258, Natural killer Group 2 member C (NKG2C), Natural killer Group 2 member D (NKG2D), B7-H3, a ligand that binds to CD83, ICAM-1, LFA-1 (CD11a/CD 18), ICOS and 4-1BB (CD137), active fragments thereof, functional derivatives thereof, and combinations thereof.

### Polynucleotide Encoding Chimeric Antigen Receptor

The present disclosure further provides polynucleotides encoding the chimeric antigen receptor polypeptides described herein. The polynucleotide encoding the CAR is prepared from an amino acid sequence of the specified CAR by any conventional method. A base sequence encoding an amino acid sequence can be obtained from the aforementioned NCBI RefSeq IDs or accession numbers of GenBenk for an amino acid sequence of each domain, and the nucleic acid of the present disclosure can be prepared using a standard molecular biological and/or chemical procedure. For example, based on the base sequence, a polynucleotide can be synthesized, and the polynucleotide of the present disclosure can be prepared by combining DNA fragments which are obtained from a cDNA library using a polymerase chain reaction (PCR). In one embodiment, the polynucleotide disclosed herein is part of a gene, or an expression or cloning cassette.

The term “polynucleotide” as used herein is defined as a chain of nucleotides. Polynucleotide includes DNA and RNA. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction (PCR), and the like, and by synthetic means.

### Polynucleotide Vector

The polynucleotide described above can be cloned into a vector. A “vector” is a composition of matter which includes an isolated polynucleotide and which can be used to deliver the isolated polynucleotide to the interior of a cell. Numerous vectors are known in the art including, but not limited to, linear polynucleotides, polynucleotides associated with ionic or amphiphilic compounds, plasmids, phagemid, cosmid, and viruses. Viruses include phages, phage derivatives. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

In one embodiment, vectors include cloning vectors, expression vectors, replication vectors, probe generation vectors, integration vectors, and sequencing vectors. In an embodiment, the vector is a viral vector. In an embodiment, the viral vector is a retroviral vector or a lentiviral vector. In an embodiment, the engineered cell is virally transduced to express the polynucleotide sequence.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art.

In one embodiment, lentivirus vectors are used. Viral vector technology is well known in the art and is described, for example, in Sambrook et al, (2001, Molecular Cloning: A Laboratory Manual, Cold Spring Harbor Laboratory, N.Y.), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Expression of chimeric antigen receptor polynucleotide may be achieved using, for example, expression vectors including, but not limited to, at least one of a SFFV or human elongation factor 11a (EF) promoter, CAG (chicken beta-actin promoter with CMV enhancer) promoter human elongation factor la (EF) promoter. Examples of less-strong/ lower-expressing promoters utilized may include, but is not limited to, the simian virus 40 (SV40) early promoter, cytomegalovirus (CMV) immediate-early promoter, Ubiquitin C (UBC) promoter, and the phosphoglycerate kinase 1 (PGK) promoter, or a part thereof. Inducible expression of chimeric antigen receptor may be achieved using, for example, a tetracycline responsive promoter, including, but not limited to, TRE3GV (Tet-response element, including all generations and preferably, the 3rd generation), inducible promoter (Clontech Laboratories, Mountain View, Calif.) or a part or a combination thereof.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor—1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure should not be limited to the use of constitutive promoters—inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionein promoter, a glucocorticoid promoter, a progesterone promoter, and a tetracycline promoter. “Expression vector” refers to a vector comprising a recombinant polynucleotide comprising expression control sequences operatively linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide,

Additional promoter elements, e.g., enhancers, regulate the frequency of transcriptional initiation. Typically, these are located in the region 30-100 bp upstream of the start site, although a number of promoters have recently been shown to contain functional elements downstream of the start site as well. The spacing between promoter elements frequently is flexible, so that promoter function is preserved when elements are inverted or moved relative to one another, in the thymidine kinase (tk) promoter, the spacing between promoter elements can be increased to 50 bp apart before activity begins to decline. Depending on the promoter, it appears that individual elements can function either cooperatively or independently to activate transcription,

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors, in other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic—resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, N.Y.). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyi phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyi phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5, 505-10). However, compositions that have different structures in solution than the normal vesicular structure are also encompassed. For example, the lipids may assume a micellar structure or merely exist as nonuniform aggregates of lipid molecules. Also contemplated are lipofectamine- nucleic acid complexes.

Regardless of the method used to introduce exogenous polynucleotides into a host cell or otherwise expose a cell to the polynucleotide of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

### Engineered Cell

In another embodiment, the disclosure provides an engineered cell expressing the chimeric antigen receptor polypeptide described above or polynucleotide encoding for the same, and described above. An “engineered cell” means any cell of any organism that is modified, transformed, or manipulated by addition or modification of a gene, a DNA or RNA sequence, or protein or polypeptide. Isolated cells, host cells, and genetically engineered cells of the present disclosure include isolated immune cells, such as NK cells and T cells that contain the DNA or RNA sequences encoding a chimeric antigen receptor or chimeric antigen receptor complex and express the chimeric receptor on the cell surface. Isolated host cells and engineered cells may be used, for example, for enhancing an NK cell activity or a T lymphocyte activity, treatment of cancer, and treatment of infectious diseases.

Any cell capable of expressing and/or capable of integrating the chimeric antigen receptor polypeptide, as disclosed herein, into its membrane may be used. In an embodiment, the engineered cell includes immunoregulatory cells. Immunoregulatory cells include T-cells, such as CD4 T-cells (Helper T-cells), CD8 T-cells (Cytotoxic T-cells, CTLs), and memory T cells or memory stem cell T cells. In another embodiment, T-cells include Natural Killer T-cells (NK T-cells). T cells comprise of CD4 and CD8 cells. CD4 is a glycoprotein present on the surface of immune cells such as T helper cells, important in T cell activation and receptor for HIV. Some monocytes or macrophages also express CD4. CD4 is also called OKT4. Cytotoxic T cells are also known as CD8+T cells or CD8 T cells expressing CD8 glycoprotein at their surfaces. These CD8+ T cells are activated once they are exposed to peptide antigens presented by MHC class I. In an embodiment, the engineered cell includes Natural Killer cells. Natural killer cells are well known in the art. In one embodiment, natural killer cells include cell lines, such as NK-92 cells. Further examples of NK cell lines include NKG, YT, NK-YS, HANK-1, YTS cells, and NKL cells. NK cells mediate anti-tumor effects without the risk of GvHD and are short-lived relative to T-cells. Accordingly, NK cells would be exhausted shortly after destroying cancer cells, decreasing the need for an inducible suicide gene on CAR constructs that would ablate the modified cells.

In one embodiment, engineered cells, in particular allogeneic T cells obtained from donors can be modified to inactivate components of TCR (T cell receptor) involved in MHC recognition. As a result, TCR deficient T cells would not cause graft versus host disease (GVHD).

### T-antigen Deficient T Cells

T cell lymphomas or T cell leukemias express specific antigens, which may represent useful targets for these diseases. For instance, T cell lymphomas or leukemias express CD5. However, CD5 are also expressed in CAR T, but not NK cells, which offset their ability of targeting these antigens. The self-killing might occur in T cells armed with CARs targeting any one of these antigens. This makes generation of CARs targeting these antigens difficult. Therefore, it may be necessary to inactivate an endogenous antigen in a T cell when it is used as a target to arm CARs.

In another embodiment, the engineered cell is further modified to inactivate cell surface polypeptide to prevent engineered cells from acting on other engineered cells. For example, the endogenous CD5, CD7 and/or CD3 gene or gene expression of the engineered cells may be knocked out or inactivated. In another preferred embodiment, the engineered cell is a T-cell having the endogenous CD5, CD7 and/or CD3 gene knocked out or inactivated. In one embodiment, the engineered cell expressing a CAR having a CD5, CD7 and/or CD3 antigen recognition domain will have the gene expressing that antigen inactivated or knocked out. For example, a T-cell having a CD5, CD7 and/or CD3 CAR will have an inactivated or knocked out CD5, CD7 and/or CD3 antigen gene. Methods to knock out or inactivate genes are known. For example, CRISPR/Cas9 system, zinc finger nuclease (ZFNs) and TALE nucleases (TALENs) and meganucleases may be used to knock out or inactivate the CD5, CD7 and/or CD3 gene or gene expression of the engineered cells.

### Sources of Cells

The engineered cells may be obtained from peripheral blood, cord blood, bone marrow, tumor infiltrating lymphocytes, lymph node tissue, or thymus tissue. The host cells may include placental cells, embryonic stem cells, induced pluripotent stem cells, or hematopoietic stem cells. The cells may be obtained from humans, monkeys, chimpanzees, dogs, cats, mice, rats, and transgenic species thereof. The cells may be obtained from established cell lines. The above cells may be obtained by any known means. The cells may be autologous, syngeneic, allogeneic, or xenogeneic to the recipient of the engineered cells.

The term “autologous” refer to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenic ally.

The term “xenogeneic” refers to a graft derived from an animal of a different species.

The term “syngeneic” refers to an extremely close genetic similarity or identity especially with respect to antigens or immunological reactions. Syngeneic systems include for example, models in which organs and cells (e.g. cancer cells and their non-cancerous counterparts) come from the same individual, and/or models in which the organs and cells come from different individual animals that are of the same inbred strain.

### Suicide System

The engineered cells of the present disclosure may also include a suicide system. Suicide systems provide a mechanism whereby the engineered cell, as described above, may be deactivated or destroyed. Such a feature allows precise therapeutic control of any treatments wherein the engineered cells are used. As used herein, a suicide system provides a mechanism by which the cell having the suicide system can be deactivated or destroyed. Suicide systems are well known in the art.

In one embodiment, a suicide system includes a gene that can be pharmacologically activated to eliminate the containing cells as required. In specific aspects, the suicide gene is not immunogenic to the host harboring the polynucleotide or cell. In one example, the suicide system includes a gene that causes CD20 to be expressed on the cell surface of the engineered cell. Accordingly, administration of rituximab may be used to destroy the engineered cell containing the gene.

In some embodiments, the suicide system includes an epitope tag. Examples of epitope tags include a c-myc tag, streptavidin-binding peptide (SBP), and truncated EGFR gene (EGFRt). In this embodiment, the epitope tag is expressed in the engineered cell. Accordingly, administration of an antibody against the epitope tag may be used to destroy the engineered cell containing the gene.

In another embodiment, the suicide system includes a gene that causes truncated epidermal growth factor receptor to be expressed on the surface of the engineered cell. Accordingly, administration of cetuximab may be used to destroy the engineered cell containing the gene. In another embodiment, the suicide gene may include caspace 8 gene, caspase 9 gene, thymidine kinase, cytosine deaminase (CD), or cytochrome P450. Examples of further suicide systems include those described by Jones et al. (Jones BS, Lamb L S, Goldman F and Di Stasi A (2014) Improving the safety of cell therapy products by suicide gene transfer. Front. Pharmacol. 5:254), which is herein incorporated by reference in its entirety.

### Engineered CRISPR Systems

Engineered CRISPR system can be used to induce genetic modifications, such as highly specific gene knockouts. CRISPR-Cas systems are native to bacteria and provide adaptive immunity against viruses and plasmids. Type-II CRISPR systems have a desirable characteristic in utilizing a single CRISPR associated (Cas) nuclease (specifically Cas9) in a complex with the appropriate guide RNAs (gRNAs). In bacteria, Cas9 guide RNAs comprise two separate RNA species: crRNA and tracrRNA. A target-specific CRISPR-activating RNA (crRNA) directs the Cas9/gRNA complex to bind and target a specific DNA sequence. The crRNA has two functional domains, a 5′-domain that is target specific and a 3′-domain that directs binding of the crRNA to the transactivating crRNA (tracrRNA). The tracrRNA is a longer, universal RNA that binds the crRNA and mediates binding of the gRNA complex to Cas9. The gRNA function can also be provided as an artificial single guide RNA (sgRNA), where the crRNA and tracrRNA are fused into a single species (see Jinek et al., Science, 337, 816-21, 2012). The sgRNA format permits transcription of a functional gRNA from a single transcription unit that can be provided by a double-stranded DNA (dsDNA) cassette containing a transcription promoter and the sgRNA sequence. In mammalian systems, these RNAs have been introduced by transfection of DNA cassettes containing RNA Pol III promoters (such as U6 or H1) driving RNA transcription, viral vectors, and single-stranded RNA following in vitro transcription (see Xu et al., Appl Environ Microbiol, 2014. 80(5):1544-52).

In the natural systems, a CRISPR associated (Cas) proteins then acts as an nuclease to cleave the targeted DNA sequence. The target sequence is identical to the guide sequence, and also contains a “protospacer-adjacent motif” (PAM) oligonucleotide adjacent and downstream (3′) to the target region in order for the system to function. Among the known Cas nucleases, such as Cas9, S. pyogenes Cas9 has been widely reported.

Cas nucleases are typically large, multi-domain proteins containing two distinct nuclease domains. Point mutations can be introduced into Cas nucleases, such as Cas9, to abolish nuclease activity, resulting in a nuclease inactive Cas nuclease, such as Cas9, that still retains its ability to bind DNA in a gRNA-programmed manner. By creating Cas nuclease, such as Cas9, fusion proteins with protein domains that alter the rate of gene translation into mRNA, e.g., transcription factors and regulators, the CRISPR-cas system functions as a RNA guided gene expression controller.

Wild-type Cas9 proteins have two functional endonuclease domains, RuvC and HNH. The RuvC domain cleaves one strand of a double strand DNA and the HNH domain cleaves another strand. When the both domains are active, the Cas9 protein can generate the DSB in genomic DNA. Cas9 proteins having only one of the enzymatic activities have been developed. Such Cas9 proteins cleave only one strand of the target DNA. For example, the RuvC and HNH domains of the Cas9 protein derived from Streptococcus pyogenes are inactivated by D10A and H840A mutations, respectively. Naturally occurring mechanism can repair double stranded or single strand nicks; however, the repairs may result in addition or deletions to the original sequences. If both of the RuvC and HNH domains of the Cas9 protein are inactivated, the Cas9 may merely sit and block transcription of the gene.

As used herein, the term “Cas nucleases, such as Cas9,” means a protein having an ability to bind to a DNA molecule in the presence of gRNA, including Cas9 proteins having both the RuvC and HNH nuclease activities and Cas9 proteins lacking either one or both of the nuclease activities. The DNA-binding activity and nuclease activity of Cas nucleases, such as Cas9, may be measured, for example, by the method described in Sternberg et al., Nature, 507, 62-67 (2014).

Cas nucleases mRNA or Cas9 mRNA may be obtained by cloning a DNA coding an amino acid sequence of a desired Cas nuclease into a vector suitable for in vitro transcription and performing in vitro transcription. Vectors suitable for in vitro transcription are known to those skilled in the art. In vitro transcription vectors that contain a cloned DNA encoding a Cas9 protein are also known and include, for example, pT7-Cas9. Methods of in vitro transcription are known to those skilled in the art.

As used herein, the term “guide RNA” or “gRNA” refers to a synthetic RNA having a fusion of a guide sequence which hybridizes to a template strand of a double stranded target sequence having a PAM that is adjacent to it on the sense sequence and “tracrRNA hybridizing segment,” e.g., segment derived from crRNA.

The tracrRNA hybridizing segment and tracrRNA may be linked together via a linker, e.g., oligonucleotide linker or otherwise. Guide RNAs generally speaking comes in different forms. One form uses separate targeting guide RNA and a tracrRNA that hybridize together to guide targeting, and another, which uses a chimeric targeting guide RNA-tracrRNA hybrid that links the two separate RNAs in a single strand of RNA that forms a hairpin, referred to as sgRNA. See also Jinek et al., Science 2012; 337:816-821.

In the natural state, crRNA is responsible for sequence specificity of gRNA. In embodiments disclosed herein, the target sequence is selected so that the sequence is present immediately upstream of a protospacer adjacent motif (PAM) in a selected double stranded nuclide acid. The target sequence may be present in either strand of the genomic DNA. However, in a preferred embodiment of this disclosure, the gRNA comprises a sequence that is identical to the sense strand that is upstream from the PAM. Tools are available for selecting a target sequence and/or designing gRNA, and lists of target sequences which are predicted for various genes in various species may be obtained. For example, Feng Zhang lab's Target Finder, Michael Boutros lab's Target Finder (E-CRISP), RGEN Tools: Cas-OFFinder, CasFinder: Flexible algorithm for identifying specific Cas9 targets in genomes, and CRISPR Optimal Target Finder, may be mentioned and the entire contents thereof are incorporated herein by reference.

Cas nucleases or Cas9 can bind to any DNA that has the PAM sequence. The exact sequence of the PAM is dependent upon the bacterial species from which the Cas nuclease or Cas9 is derived. One Cas9 protein is derived from Streptococcus pyogenes and the corresponding PAM sequence is NGG (SEQ ID NO: 4) present immediately downstream of the 3′ end of the target sequence, wherein N represents any one of A, T/U, G, and C. PAM sequences of various bacterial species are known.

In bacteria, tracrRNA hybridizes to a part of gRNA to form a hairpin loop structure. The structure is recognized by Cas9 protein and a complex of crRNA, tracrRNA and Cas9 protein is formed. Thus, tracrRNA is responsible for the ability of gRNA to bind to Cas9 protein. tracrRNA is derived from an endogenous bacterial RNA and has a sequence intrinsic to the bacterial species. tracrRNA derived from the bacterial species known to have a CRISPR system listed above may be used herein. Preferably, tracrRNA and Cas9 protein derived from the same species are used.

gRNA may be obtained by cloning a DNA having a desired gRNA sequence into a vector suitable for in vitro transcription and performing in vitro transcription. Vectors suitable for in vitro transcription are known to those skilled in the art. In vitro transcription vectors that comprise a sequence corresponding to gRNA with no target sequence are also known in the art. gRNA may be obtained by inserting a synthesized oligonucleotide of a target sequence into such vector and performing in vitro transcription. Such vectors include, for example, pUC57-sgRNA expression vector, pCFD1-dU6: 1gRNA, pCFD2-dU6:2gRNA pCFD3-dU6:3gRNA, pCFD4-U6:1 U6:3tandemgRNAs, pRB17, pMB60, DR274, SP6-sgRNA-scaffold, pT7-gRNA, DR274, and pUC57-Simple-gRNA backbone available from Addgene, and pT7-Guide-IVT available from Origene. Methods of in vitro transcription are known to those skilled in the art.

### Method of Making Engineered Cells

In one embodiment, the disclosure also provides methods of making the engineered cells described above. In this embodiment, the cells described above are obtained or isolated. The cells may be isolated by any known means. The cells include peripheral blood cells or cord blood cells. In another embodiment, the cells are placental cells, embryonic stem cells, induced pluripotent stem cells, or hematopoietic stem cells.

The polynucleotide encoding for the chimeric antigen receptor polypeptide described above is introduced into the peripheral blood cells or cord blood cells by any known means. In one example, the polynucleotide encoding for the chimeric antigen receptor polypeptide described above is introduced into the cell by way of viral vector.

The polynucleotide encoding for the chimeric antigen receptor polypeptide described above is introduced into the placental cells, embryonic stem cells, induced pluripotent stem cells, or hematopoietic stem cells by any known means. In one example, the polynucleotide encoding for the chimeric antigen receptor polypeptide described above is introduced into the cell by way of viral vector.

In other embodiments, the chimeric antigen receptor polynucleotide may be constructed as a transient RNA-modified “biodegradable derivatives”. The RNA-modified derivatives may be electroporated into a T cell or NK cell. In a further embodiment, chimeric antigen receptor described herein may be constructed in a transposon system also called a “Sleeping Beauty”, which integrates the chimeric antigen receptor polynucleotide into the host genome without a viral vector.

Once the polynucleotide described above is introduced into the cell to provide an engineered cell, the engineered cells are expanded. The engineered cells containing the polynucleotide described above are expanded by any known means. The expanded cells are isolated by any known means to provide isolated engineered cells according to the present disclosure.

### Methods of Using

The disclosure provides methods to kill, reduce the number of, or deplete immunoregulatory cells. In another embodiment, the disclosure provides a method to kill, reduce the number of, or deplete cells having CD5, CD7 and/or CD3. As used herein, “reduce the number of includes a reduction by at least 5%, at least 10%, at least 25%, at least 50%, at least 75%, at least 80%, at least 90%, at least 99%, or 100%. As used herein, “deplete” includes a reduction by at least 75%, at least 80%, at least 90%, at least 99%, or 100%.

In one embodiment, the disclosure includes a method of reducing the number of immunoregulatory cells having CD5, CD7 and/or CD3 by contacting the immunoregulatory cells with an effective amount of the engineered cells described above expressing a chimeric antigen receptor peptide having a CD5, CD7 and/or CD3 antigen recognition domain. Optionally, the reduction in the number of immunoregulatory cells having CD5, CD7 and/or CD3 may be determined by any cell assay known in the art.

As used herein, the immunoregulatory cells may be in a patient, in cell culture, or isolated. As used herein, “patient” includes mammals. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order. Anthropoids (humans and apes). Preferably, the mammal is a human.

In certain embodiments, the patient is a human 0 to 6 months old, 6 to 12 months old, 1 to 5 years old, 5 to 10 years old, 5 to 12 years old, 10 to 15 years old, 15 to 20 years old, 13 to 19 years old, 20 to 25 years old, 25 to 30 years old, 20 to 65 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old.

The terms “effective amount” and “therapeutically effective amount” of an engineered cell as used herein mean a sufficient amount of the engineered cell to provide the desired therapeutic or physiological or effect or outcome. Such, an effect or outcome includes reduction or amelioration of the symptoms of cellular disease. Undesirable effects, e.g. side effects, are sometimes manifested along with the desired therapeutic effect; hence, a practitioner balances the potential benefits against the potential risks in determining what an appropriate “effective amount” is. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. Generally, the engineered cell or engineered cells is/are given in an amount and under conditions sufficient to reduce proliferation of target cells.

In one embodiment, the disclosure includes a method of reducing the number of immunoregulatory cells having a T-cell antigen such as CD5, CD7 and/or CD3 by contacting the immunoregulatory cells with an effective amount of the engineered cells described above expressing a chimeric antigen receptor peptide having a T-cell antigen antigen recognition domain. Optionally, the reduction in the number of immunoregulatory cells having T-cell antigen may be determined by any cell assay known in the art.

### Method of Treatment

In another embodiment, the disclosure provides methods for the treatment of a cell proliferative disease. The method includes administration of a therapeutically effective amount of the engineered cells described above to a patient in need thereof. Cell proliferative disease is any one of cancer, neoplastic disease or any disease involving uncontrolled cell proliferation (e.g. formation of cell mass) without any differentiation of those cells into specialized and different cells. Cell proliferative diseases as also include a malignancy, or a precancerous condition such as a myelodysplasia syndrome or a preleukemia, or prelymphoma. With respect to the disclosed methods, the cancer can be any cancer, including any of acute lymphocytic cancer, acute myeloid leukemia, alveolar rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma), lymphoma, malignant mesothelioma, mastocytoma, melanoma, multiple myeloma, nasopharynx cancer, non-Hodgkin lymphoma, B-chronic lymphocytic leukemia, hairy cell leukemia, acute lymphoblastic leukemia (ALL), T-cell acute lymphocytic leukemia, and Burkitt's lymphoma, extranodal NK/T cell lymphoma, NK cell leukemia/lymphoma, post-transplant lymphoproliferative disorders, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, and ureter cancer. Preferably, the cancer is a hematological malignancy (e.g., leukemia or lymphoma, including but not limited to Hodgkin lymphoma, non-Hodgkin lymphoma, chronic lymphocytic leukemia, acute lymphocytic cancer, acute myeloid leukemia, B-chronic lymphocytic leukemia, hairy cell leukemia, acute lymphoblastic leukemia (ALL), and Burkitt's lymphoma), thymic carcinoma, diffuse large cell lymphoma, mantle cell lymphoma, small lymphocytic lymphoma (SLL), and chronic lymphoid leukemia(CLL), T-cell lymphoma, and peripheral T-cell lymphoma.

The disclosure provides a method for the treatment of acute organ rejection by depletion of T cells that are associated with a T-cell antigen such as CD5, CD7 and/or CD3.

In one embodiment, the disclosure incudes a method for the treatment of acute or chronic graft versus host disease (GVHD) by depletion of T cells that are associated with at least one of a T-cell antigen such as CD5, CD7, or CD3.

In one embodiment, the disclosure incudes a method for the depletion or reduction of donor and host T cells using CAR T cells in vivo for stem cell transplant. This could be accomplished by administration of CAR T cells to a patient immediately before the infusion of the bone marrow stem cell graft.

The disclosure provides a method of immunotherapy as a conditioning or bridge-to-transplant strategy or stand-alone for the treatment of cell proliferative diseases that are associated with a T-cell antigen such as CD5, CD7 and/or CD3.

The disclosure provides a method for the treatment of cell proliferative diseases that are associated with a T-cell antigen such as CD5, CD7 and/or CD3.

In another embodiment, the disclosure provides a method for the treatment of non-cancer related diseases that are associated with the expression of a T-cell antigen such as CD5, CD7 and/or CD3.

In some embodiments, CAR having a T-cell antigen recognition domain for use in the treatment of a cell proliferative disease is combined with another anticancer agent. In certain embodiments, the anti-cancer agent selected from abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelali sib, imatinib, talimogene laherparepvec, ipilimumab, romidepsin, ixabepilone, ixazomib, ruxolitinib, cabazitaxel, palifermin, pembrolizumab, ribociclib, ti sagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof.

In some embodiments, CAR having a T-cell antigen, e.g., CD5, CD7 and/or CD3 antigen recognition domain for use in the treatment of a cell proliferative disease is combined with a checkpoint blockade, such as CTLA-4 and PD1/PD-Ll. In certain embodiments, the chemotherapy agent is an anti-PD-1, anti-CTLA4 antibody or combinations thereof, such as an anti-CTLA4 (e.g., ipilimumab, tremelimumab) and anti-PD1 (e.g., nivolumab, pembrolizumab, atezolizumab, avelumab, durvalumab). In certain embodiments, the method of administration is in a subject with a lymphodepleted environment. In certain embodiments, lymphodepleting agents are, e.g., cyclophosphamide and fludarabine.

In some embodiments, CARs having a T-cell antigen, e.g., CD5, CD7 and/or CD3 antigen recognition domain are used as a strategy to deepen, remove, reduce, resist and/or prolong responses to initial chemotherapy, or when combined with other adjunct therapies. All available adjunct therapies to treat or prevent the disease condition are considered to be part of this disclosure and are within the scope of the present disclosure

In another embodiment, administration of a CAR polypeptide having a T-cell antigen, e.g., CD5, CD7 and/or CD3 antigen recognition domain is used to treat rheumatoid arthritis. In another embodiment, T-cell antigen, e.g., CD5, CD7 and/or CD3 -CAR may be used as a prophylaxis for graft-versus-host disease following bone marrow transplantation therapy (BMT) therapy. In another embodiment, T-cell antigen, e.g., CD5, CD7 and/or CD3-CAR may be used to modify of expression in treatment of autoimmune disorders and malignancies.

In some embodiments, the disclosure of engineered cell having a chimeric antigen receptor selective for CD5 may act as a bridge to bone marrow transplant for those patients who are not longer responding to chemotherapy or have minimal residual diseases and are not eligible for bone marrow transplant.

In particular embodiments, CD5, CD7 and/or CD3-CAR a T cell targets cells that express CD5, CD7 and/or CD3. Target cells may be, but is not limited to cancer cells, such as T-cell lymphoma or T-cell leukemia, precursor acute T-cell lymphoblastic leukemia/lymphoma, B cell chronic lymphocytic leukemia/small lymphocytic lymphoma, mantle cell lymphoma, CD5, CD7 and/or CD3 positive diffuse large B cell lymphoma, and thymic carcinoma.

In one embodiment, CD5, CD7 and/or CD3-CAR may be used for treating non-hematologic disorders including, but not limited to, rheumatoid arthritis, graft-versus-host-disease and autoimmune diseases.

The engineered or modified T cells may be expanded in the presence of IL-2 or/and both IL-7 and IL-15, or using other molecules.

The introduction of CARs can be fulfilled before or after the inactivation of CD5, CD7 and/or CD3 by expanding in vitro engineered T cells prior to administration to a patient.

In some embodiments, CD5, CD7 and/or CD3 targeted CAR T cells are co-administrated with immunomodulatory drugs, such as, but not limited to CTLA-4 and PD-1/PD-L1 blockades, or cytokines, such as IL-2 and IL12 or inhibitors of colony stimulating factor-1 receptor (CSF1R), such as FPA008.

In another embodiment, the disclosure provides a method of imparting, aiding, increasing, or boosting anti-leukemia or anti-lymphoma immunity.

The therapeutic agent including the engineered cell expressing the CAR as an active ingredient can be administered intradermally, intramuscularly, subcutaneously, intraperitoneally, intranasally, intraarterially, intravenously, intratumorally, or into an afferent lymph vessel, by parenteral administration, for example, by injection or infusion, although the administration route is not limited.

Any method of the disclosure may further includes the step of delivering to the individual an additional cancer therapy, such as surgery, radiation, hormone therapy, chemotherapy, immunotherapy, or a combination thereof. Chemotherapy includes, but is not limited to, CHOP (cyclophosphamide, doxorubicin, vincristie, prednisone), EPOCH (etoposide, vincristine, doxorubicin, cyclophosphamide, prednisone), or any other multidrug regimens. In a preferred embodiment, CD54 targeted CAR cells are utilized for treating or preventing a residual disease after stem cell transplant and/or chemotherapy.

In another embodiment, any method of the disclosure may further include antiviral therapy, cidofovir and interleukin-2, Cytarabine (also known as ARA-C) or natalizumab treatment for MS patients or efalizumab treatment for psoriasis patients or other treatments for PML patients. In further aspects, the T cells of the disclosure may be used in a treatment regimen in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation. Drugs that inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993) can also be used. In a further aspect, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In one aspect, the cell compositions of the present disclosure are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present disclosure. In an additional embodiment, expanded cells are administered before or following surgery.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia greata, ankylosing spondylitis, autoimmune hepatitis, autoimmune parotitis, Crohn's disease, diabetes (Type 1), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, and ulcerative colitis.

The present disclosure may be better understood with reference to the examples, set forth below. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Following administration of the delivery system for treating, inhibiting, or preventing a cancer, the efficacy of the therapeutic engineered cell can be assessed in various ways well known to the skilled practitioner. For instance, a therapeutic engineered cell delivered in conjunction with the chemo-adjuvant is efficacious in treating or inhibiting a cancer in a subject by observing that the therapeutic engineered cell reduces the cancer cell load or prevents a further increase in cancer cell load. Cancer cell loads can be measured by methods that are known in the art, for example, using polymerase chain reaction assays to detect the presence of certain cancer cell nucleic acids or identification of certain cancer cell markers in the blood using, for example, an antibody assay to detect the presence of the markers in a sample (e.g., but not limited to, blood) from a subject or patient, or by measuring the level of circulating cancer cell antibody levels in the patient.

### Development of Chimeric Antigen Receptors Targeting T-Cell Malignancies Using Two Structurally Different Anti-CD5 Antigen Binding Domains in NK and CRISPR-Edited T Cell Lines

Patients with relapsed T-cell acute lymphoblastic leukemia or lymphoblastic lymphoma (T-ALL/T-LLy) have dismal outcomes, with mortality rates greater than 80%, when treated with chemotherapy alone. Allogeneic hematopoietic stem cell transplantation (HSCT) offers the greatest chance of cure in these patients. A recent study by the Center for International Blood and Marrow Transplant Research showed the 3-year overall survival (OS) with HSCT is 48% for patients able to achieve complete second remission (CR2) prior to transplantation. For patients with first relapse of T-ALL/LLy, achieving CR2 is the most important step prior to HSCT, as disease status at the time of transplantation remains the most important factor associated with overall survival. However, attaining clinical remission after relapse remains the biggest therapeutic challenge in T-cell disease, and most patients are unable to receive transplantation given the aggressive nature of relapsed disease. Thus, in order to maximize and improve upon the benefits of an allogeneic HSCT, there remains a need to develop newer strategies to induce remission in these relapsed patients.

CAR-based immunotherapy can play an important role by providing a sustained remission post-relapse, thereby acting as a bridge to stem cell transplantation. Unlike CAR therapy in B-cell malignancies, where sustained B-cell aplasia due to off-target toxicity can be managed with periodic intravenous immunoglobulin infusions, persistent T-cell aplasia caused by T-cell-directed CAR therapy would result in life threatening severe immunosuppression. Thus, hematopoietic stem cell transplantation (HSCT) to allow for immune reconstitution following CAR T cell therapy is a reasonable strategy.

Cytotoxicity and T-cell activation was demonstrated using an anti-CD5-VLR-CAR. Using CD5-CAR T cells with CRISPR-Cas9 genome editing is an approach to prevent fratricide. Self-activation of CD5-positive CD5-CAR-modified effector cells occurs due to interactions with self and neighboring CD5 antigens. Tests using both scFv- and VLR-based CD5-CARs indicate that this effect diminished over time as the average number of transgene copies per cell decreased. One approach to prevent effector cell activation in the absence of malignant cells is to use CD5-negative NK cells modified to express the anti-CD5 CARs. In vitro and in vivo data indicate that NK-92 cells modified to express CD5-CARs are effective in targeting a CD5-positive T-cell leukemia cell line. It is contemplated that persistence of NK-92 cells, effector cell, optionally in combination with IL-2, may include repeated dosing or by transitioning to primary NK CD5-CAR cells to enhanced anti-tumor efficacy in a T-cell leukemia mouse model.

Another approach is to knock out the target antigen from the effector cells using genome-editing. CD5-edited CD5-CAR-modified Jurkat T cells exhibit decreased self-activation, yet increased activation when cultured with target cells. CD5-edited effector cells were significantly more activated when in culture with target T cells compared to their initial levels of activation in culture alone. CD5-CAR expression in T cells results in down-regulation of CD5. Interestingly, data reported herein indicates that non-edited CAR-modified T cells have decreased CD5-CAR protein expression compared to CD5-edited CAR-modified T cells. This data is shown in both Jurkat T cells and primary T cells. Furthermore, in cultures with CD5-edited effector cells and target cells, effector cells interact more robustly with CD5 on target cells; whereas CD5-positive, non-edited effector T cells interact with CD5 antigen on both effector and target cells, reducing their potency. Overall, the data shows CD5-negative effector cells are advantageous compared to CD5-positive effector cells due to their decreased self-activation and increased CAR expression.

CD5-edited effector cells have a greater effect on target cell CD5 expression. The CD5-VLR used in the CAR construct is an avidity-based antibody, with the multimeric form of the VLR antibody binding to human CD5 with a higher efficiency compared to the monomeric form. The scFv was derived from the murine H65 anti-human CD5 IgG antibody. It cannot be concluded from in vitro studies which CD5-CAR would be most advantageous, as both demonstrate substantial target cell association and effector cell activation. However, the in vivo studies indicated the VLR-CAR did not perform as well as the scFv-CAR.

NK-cells as effector cells and CD5 knockout in effector T cells, modified with the CD5-CARs have the potential to overcome the barriers of self-activation and fratricide, which are issues that are hampering the use of CAR therapies from being applied to the treatment of T-cell malignancies. One object of this disclosure is to provide a bridge to allogenic transplantation for relapsed patients. Strategies using CAR modified immunocompetent cells also are contemplated as therapeutics to attain long-term remission in these patients.

### EXAMPLES

### Construction of CD5-Directed CARs

A CD5-VLR-CAR was generated using a VLR protein sequence shown to be specific for the CD5 antigen. The sequence for the CD5-scFv was generated using a published humanized murine immunoglobulin protein sequence. Studnicka et al., Human-engineered monoclonal antibodies retain full specific binding activity by preserving non-CDR complementarity-modulating residues. Protein Eng. 1994, 7(6):805-14. The cDNA sequence designed to express the scFv was codon optimized for human cell expression. The C-terminus of VH was joined with the N-terminus of VL using a 15 bp linker encoding a glycine and serine pentapeptide repeat (G4S)3.

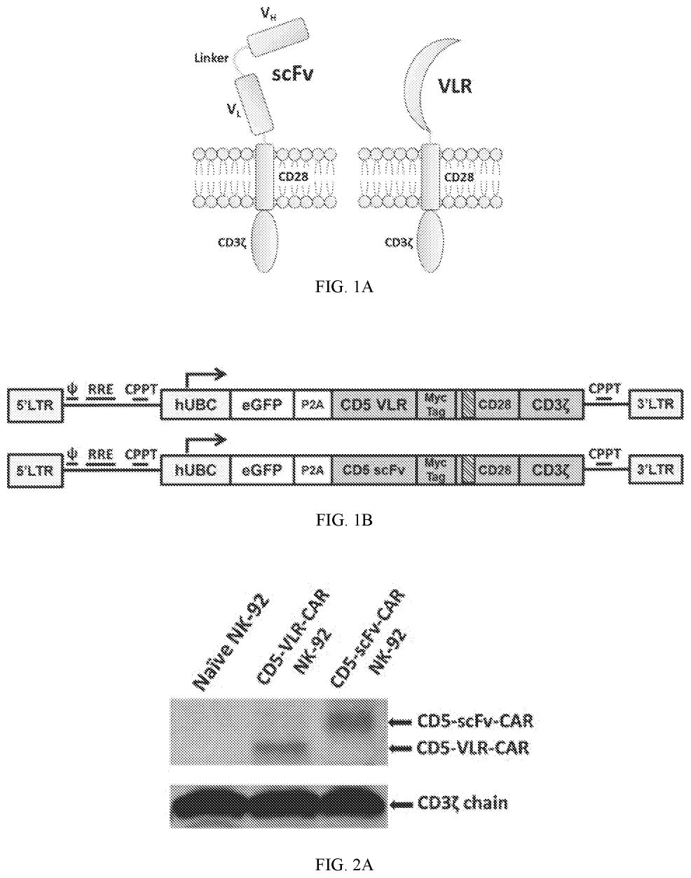

The entire CD5-scFv sequence totaled 720 bp compared to the shorter 570 bp CD5-VLR sequence. The two CD5 sequences were cloned into the CAR cassette, which is a second generation CAR composed of an N-terminal IL-2 signal peptide followed by the CD5-VLR or -scFV antigen binding domain, the transmembrane and intracellular domains of CD28, and the intracellular signaling domain of CD3zeta (FIG. 1A). A bicistronic vector co-expressing eGFP and the CD5-CAR via a self-cleaving 2A peptide sequence (P2A) was used to enable selection of positively transduced cells by flow sorting (FIG. 1B).

CD5 VLR CAR plasmid sequence: N-terminal IL-2
signal sequence followed by CD5 VLR (bold),
CD28 (bold), and CD3zeta
(SEQ ID NO: 6)
ATGTACAGGATGCAACTCCGTCTTGCATTGCACTAAGTCTTGCACTTGTC
ACGAATTCGGGCGCGCCTTGTCCTTCACAGTGCTCCTGCAGCGGAACCGA
GGTCCATTGTCAGAGAAAATCCCTGGCTTCAGTCCCTGCCGGAATCCCAA
CCACAACAAGGGTGCTGTACCTGCACGTCAACGAGATTACTAAGTTCGAA
CCAGGAGTGTTTGACCGCCTGGTCAACCTGCAGCAGCTGTATCTGGGAGG
AAATCAGCTGAGCGCCCTGCCAGACGGCGTGTTCGATCGACTGACTCAGC
TGACCAGACTGGATCTGTACAACAATCAGCTGACCGTGCTGCCTGCCGGG
GTCTTTGACCGACTGGTGAATCTGCAGACACTGGATCTGCACAACAATCA
GCTGAAGTCTATCCCCAGAGGCGCATTCGACAACCTGAAAAGTCTGACCC
ATATTTGGCTGTTTGGGAATCCTTGGGACTGCGCCTGTAGCGATATCCTG
TATCTGTCCGGATGGCTGGGACAGCATGCAGGGAAAGAGCAGGGACAGGC
TGTCTGCTCTGGCACCAACACACCCGTGCGGGCTGTCACCGAGGCATCAA
CATCCCCATCAAAGTGTCCTGGCTACGTGGCAACAACCAGATCTGCTAGC
GAGCAGAAGCTGATCAGCGAGGAGGACCTGGACAATGAGAAGAGCAATGG
AACCATTATCCATGTGAAAGGGAAACACCTTTGTCCAAGTCCCCTATTTC
CCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAG
GAGTAAGAGGAGCAGGCTCCTGCACAGTGACTACATGAACATGACTCCCA
GGAGGCCTGGGCCAACCCGCAAGCATTACCAGCCCTATGCCCCACCACGC
GACTTCGCAGCCTATCGCTCCAGCAGGAGCGCAGACGCTCCCGCGTACCA
GCAGGGCCAGAACCAGCTCTATAACGAGCTCAATCTAGGACGAAGAGAGG
AGTACGATGTTTTGGACAAGAGACGTGGCCGGGACCCTGAGATGGGAGGC
AAGCCGAGAAGGAAGAACCCTCAGGAAGGCCTGTACAATGAACTGCAGAA
AGATAAGATGGCGGAGGCCTACAGTGAGATTGGGATGAAAGGCGAGCGCC
GGAGGGGCAAGGGGCACGATGGCCTTTACCAGGGTCTCAGTACAGCCACC
AAGGACACCTACGACGCCCTTCACATGCAGGCCCTGCCTCCTCGCTGA
CD5 scFv CAR plasmid sequence: IL-2 signal
sequence followed by CD5 scFv(bold), CD28
(bold), and CD3zeta
(SEQ ID NO: 7)
ATGTACAGGATGCAACTCCTGTCTTGCATTGCACTAAGTCTTGCACTTGT
CACGAATTCGGGCGCGCCTGAAATTCAGTTGGTGCAAAGCGGAGGTGGCC
TTGTGAAGCCAGGAGGCAGTGTGCGAATTAGTTGTGCAGCCTCCGGTTAC
ACGTTCACCAACTATGGCATGAACTGGGTGAGACAGGCCCCCGGCAAGGG
GTTGGAATGGATGGGCTGGATTAACACACATACGGGCGAACCGACATACG
CCGACAGCTTTAAAGGTCGATTTACTTTTAGCTTGGACGATTCCAAAAAT
ACGGCATACCTGCAAATAAACTCACTGCGGGCAGAGGATACGGCCGTATA
TTTTTGTACGCGGAGAGGGTACGATTGGTACTTTGATGTCTGGGGACAGG
GGACGACAGTAACCGTGTCTAGTGGCGGGGGAGGATCAGGTGGTGGCGGT
AGCGGTGGAGGTGGAAGTGATATCCAGATGACACAATCACCGAGTTCCCT
GTCCGCGTCAGTAGGGGATCGGGTGACAATTACATGTAGAGCATCTCAAG
ACATCAATAGCTACCTGAGCTGGTTTCAGCAAAAGCCCGGAAAAGCTCCG
AAAACTCTGATTTATCGGGCCAATCGCCTTGAGTCTGGGGTGCCAAGTAG
ATTTTCAGGCTCCGGGAGCGGGACGGACTATACGTTGACCATATCAAGTC
TTCAGTACGAGGACTTCGGGATATACTATTGCCAACAGTACGATGAGAGC
CCGTGGACCTTCGGGGGTGGGACAAAGTTGGAGATCAAAGCTAGCGAGCA
GAAGCTGATCAGCGAGGAGGACCTGGACAATGAGAAGAGCAATGGAACCA
TTATCCATGTGAAAGGGAAACACCTTTGTCCAAGTCCCCTATTTCCCGGA
CCTTCTAAGCCCTTTTGGGTGCTGGTGGTGGTTGGTGGAGTCCTGGCTTG
CTATAGCTTGCTAGTAACAGTGGCCTTTATTATTTTCTGGGTGAGGAGTA
AGAGGAGCAGGCTCCTGCACAGTGACTACATGAACATGACTCCCAGGAGG
CCTGGGCCAACCCGCAAGCATTACCAGCCCTATGCCCCACCACGCGACTT
CGCAGCCTATCGCTCAGCAGGAGCGCAGACGCTCCCGCGTACCAGCAGGG
CCAGAACCAGCTCTATAACGAGCTCAATCTAGGACGAAGAGAGGAGTACG
ATGTTTTGGACAAGAGACGTGGCCGGGACCCTGAGATGGGAGGCAAGCCG
AGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGG
GCAAGGGGCACGATGGCCTTTACCAGGGTCTCAGTACAGCCACCAAGGAC
ACCTACGACGCCCTTCACATGCAGGCCCTGCCTCCTCGC
CD5 scFv (CDRs are bold)
(SEQ ID NO: 8)
EIQLVQSGGGLVKPGGSVRISCAASGYTFTNYGMNWVRQAPGKGLEWMGW
INTHTGEPTYADSFKGRFTFSLDDSKNTAYLQINSLRAEDTAVYFCTRRG
YDWYFDVWGQGTTVTVSSGGGGSGGGGSGGGGSDIQMTQSPSSLSASVGD
RVTITCRASQDINSYLSWFQQKPGKAPKTLIYRANRLESGVPSRFSGSGS
GTDYTLTISSLQYEDFGIYYCQQYDESPWTFGGGTKLEIK
CD5 signal peptide (start of CD5 translation)
PAM and CD5 target sequence (bold)
(SEQ ID NO: 10)
ACCATGCCCATGGGGTCTCTGCAACCGCTGGCCACCTTGTACCTGCTGGG
GATGCTGGTCGCTTCCTGCCTCGGACGGCT. . .
CRISPR guide RNA and TracrRNA sequence #2:
sequence include the U6 Promoter followed by
the CD5 target gRNA (bold), and TracrRNA-
targets hybridization to the coding strand
(SEQ ID NO: 9)
GACTCTTCGCGATGTACGGGCCAGATATACGCGTAAGGTCGGGCAGGAAG
AGGGCCTATTTCCCATGATTCCTTCATATTTGCATATACGATACAAGGCT
GTTAGAGAGATAATTAGAATTAATTTGACTGTAAACACAAAGATATTAGT
ACAAAATACGTGACGTAGAAAGTAATAATTTCTTGGGTAGTTTGCAGTTT
TAAAATTATGTTTTAAAATGGACTATCATATGCTTACCGTAACTTGAAAG
TATTTCGATTTCTTGGCTTTATATATCTTGTGGAAAGGACGAAACACCGA
GCGGTTGCAGAGACCCCATGTTTTAGAGCTAGAAATAGCAAGTTAAAATA
AGGCTAGTCCGTTATCAACTTGAAAAAGTGGCACCGAGTCGGTGCTTTTT
T

### CD5-CAR NK-Cell Mediated Cytotoxicity

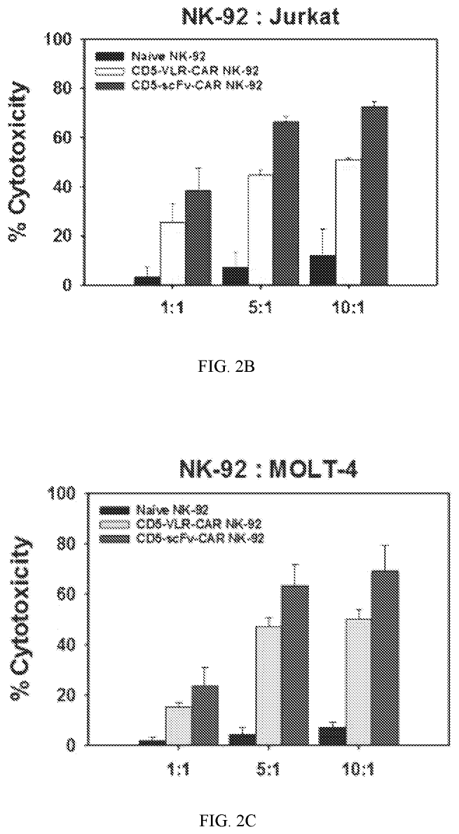

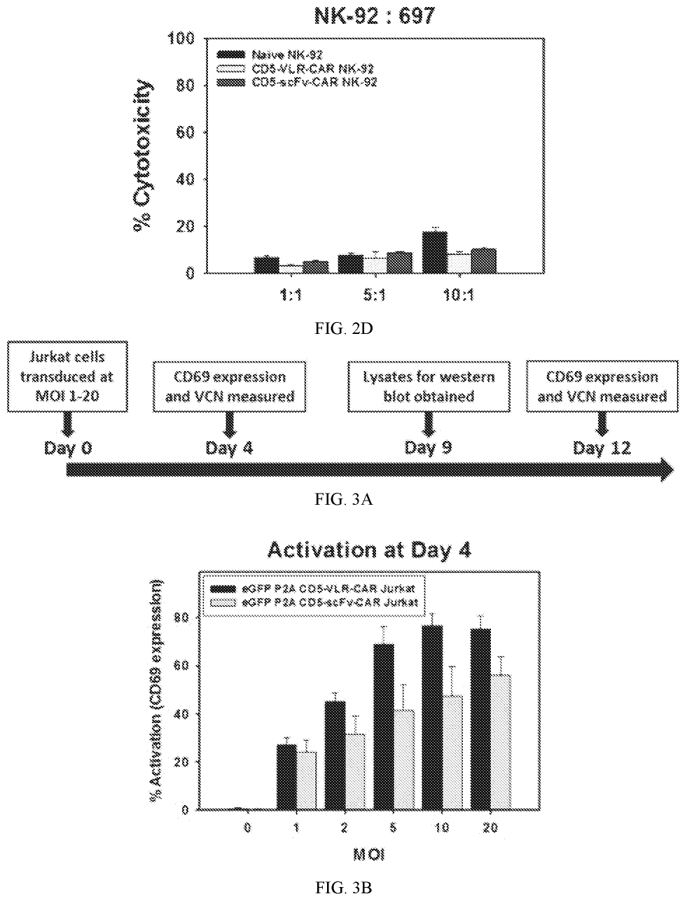

To demonstrate CAR-directed cytotoxicity, the well-characterized cytotoxic human NK cell line, NK-92, was used, which is an interleukin-2 (IL-2) dependent immortalized cell line that has maintained its cytotoxic capabilities. NK-92 cells do not display CD5 on their surface, and this allows for expression of the CD5-CAR without self-activation and fratricidal killing of transduced cells. To generate CD5-scFv-CAR expressing NK-92 cells, they were transduced with the bicistronic construct expressing eGFP and the CD5-scFv-CAR. Poor transduction efficiency (<5%) was observed after the initial lentiviral vector transduction. As with the CD5-VLR-CAR-expressing NK-92 cells, flow sorting was used to generate a CD5-scFv-CAR expressing NK-92 cell line using eGFP as a selection marker for positively transduced cells. After two rounds of flow sorting for eGFP, a CD5-scFv-CAR expressing NK-92 population was generated with 99% eGFP expression. qPCR analysis demonstrated an average of 1.0 transduced gene copy/cell in the sorted and expanded cells. To confirm CD5-CAR expression in the flow sorted NK-92 cell lines, western blot analysis was performed using a CD3zeta antibody. Bands of 48 and 55 kDa were visible corresponding to the CD5-VLR-CAR and CD5-scFv-CAR proteins respectively (FIG. 2A).

To assess their cytotoxic potential, CD5-CAR expressing NK-92 effector (E) cells were cultured with CD5-positive Jurkat and MOLT-4 T-cell leukemia target (T) cells at varying E:T ratios. The CD5-negative B-cell leukemia cell line, 697, was used as a negative control. The target cells were pre-labeled with the membrane dye PKH26, which allowed for easy distinction from the non-labeled effector cells using flow cytometry. Cytotoxicity was measured via uptake of 7-AAD, a marker for cell death, into target cells. A significant increase in cytotoxicity was observed with the CD5-CAR expressing NK-92 cells compared to Naive NK-92 cells, even at the lowest E:T ratios (FIG. 2B and FIG. 2C). Greater cytotoxicity was observed in the CD5-scFv-CAR group at the higher E:T ratios, however, the difference in cytotoxicity was not significant between the VLR-CAR and scFv-CAR at the lower 1:1 E:T ratio. No increase in cytotoxicity was seen when the CD5-CAR NK-92 cells were tested against the CD5-negative 697 cell line (FIG. 2D).

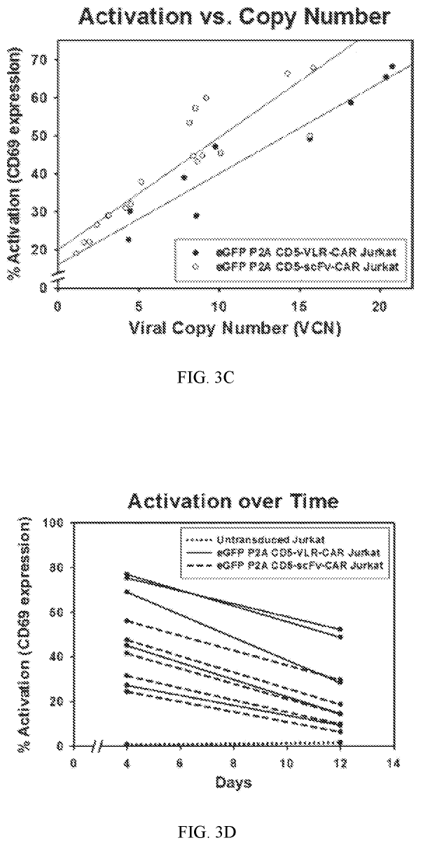

CD5 CAR-Directed T-Cell Activation In order to analyze the effect of CD5-CARs on T cells, the CD5-positive Jurkat T-cell leukemia line was transduced with the lentiviral vector encoding eGFP and a CD5-CAR at MOIs ranging from 1 to 20. To measure T-cell activation induced by engagement of CD5-CARs with CD5 on neighboring cells, surface expression of the T-cell activation marker, CD69, was measured by flow cytometry 4 and 12 d after transduction (FIG. 3A). The degree of activation correlated with the transduction vector amount, with increasing activation in a dose dependent manner. Higher activation was observed in the CD5-VLR-CAR expressing Jurkat T cells compared to those expressing the CD5-scFv- CAR, and no activation was observed in eGFP negative cells.

To confirm integration of the CD5-CAR transgene into the Jurkat T-cell genome, proviral vector copy number (VCN) was measured using quantitative PCR. Increases in VCN were correlated with increases in vector amount and increases in activation (FIG. 3C). The CD5-VLR-CAR Jurkat T cells had a higher VCN compared to the CD5-scFv-CAR cells at corresponding MOIs, which is likely the reason for the slightly higher activation observed in the CD5-VLR-CAR cells (FIG. 3B). When comparing the activation between the two CD5-CAR-modified cell populations as a function of VCN, a linear correlation was found in both groups (R2=0.91 for CD5-VLR-CAR, R2=0.82 for CD5-scFv-CAR) and the CD5-scFv-CAR cells exhibited higher activation compared to the CD5-VLR-CAR cells (FIG. 3C). As a means of measuring CD5-CAR protein expression in the transduced T cells, Western blot analysis was performed on whole cell lysates 9 d after transduction. CD5-CAR proteins were detected using an anti-CD3zeta antibody. Proteins of approximately 48 and 55 kDa were observed, which corresponded to the predicted sizes of the CD5-VLR-CAR and CD5-scFv-CAR, respectively, as well as an 18 kDa band, which corresponded to the molecular weight of the endogenous CD3zeta protein known to be expressed in Jurkat T cells. CAR expression increased in a vector MOI dependent manner. On day 12 post-transduction, activation and VCN were measured again in both CD5-CAR-expressing Jurkat T-cell populations. A decrease in VCN from day 4 to day 12 was observed, as was a corresponding decrease in CD69 expression (FIG. 3D). Although this decrease in Jurkat T-cell activation and VCN can, in part, be due to pseudo transduction, it also likely results from the faster proliferation rate of non-modified cells compared to CD5-CAR expressing cells, as well as from activation induced cell death resulting from continuous activation of the transduced cell population through interactions with CD5 antigen on self and neighboring cells.

### CD5 Knockout in Jurkat T Cells Using CRISPR-Cas9 Genome Editing

To increase the effectiveness of anti-CD5-directed CAR T cells, CD5 expression was knocked out in Jurkat T cells using CRISPR-Cas9 genome editing. In T cells, only full-length CD5 protein is expressed. However, in CD5-positive B cells, alternative splicing of exon 1 results in an alternate exon, termed exon 1B, that encodes a truncated, cytosolic CD5 protein. Targeting sequences early in the gene, upstream of the splice site, may generate a non-functional protein product and avoid the alternative splicing event. Although T cells do not express exon 1B naturally, a balance between the expression of exon 1A and exon 1B has been implicated in T cells, which may occur if exons downstream to 1A are edited. Three gRNAs where generated with different targeting sequences within the first 100 bp of exon

1A to knockout CD5 expression. Each gRNA was expressed in conjunction with Cas9, derived from Streptococcus pyogenes, on a single plasmid.

Gene knockout with CRISPR technology may be accomplished by Cas9-mediated dsDNA or ssDNA breaks. After a break or nick, natural repair mechanisms, such as non-homologous end joining (NHEJ), frequently leads to deletions and insertions resulting frameshifts disrupt the transcription of the altered sequence. When using S. pyogenes Cas9, potential target sites are both [5′-20nt-NGG-3′] and [5′ -CCN-20nt-3], where N is any nucleotide. Thus, one can target the coding or template strand of DNA.

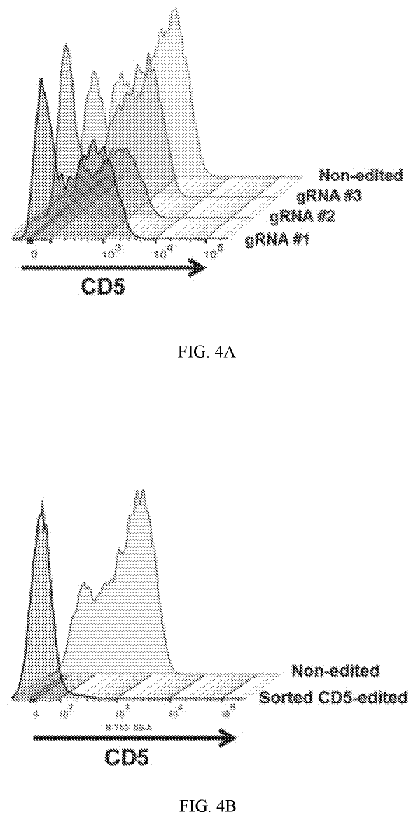

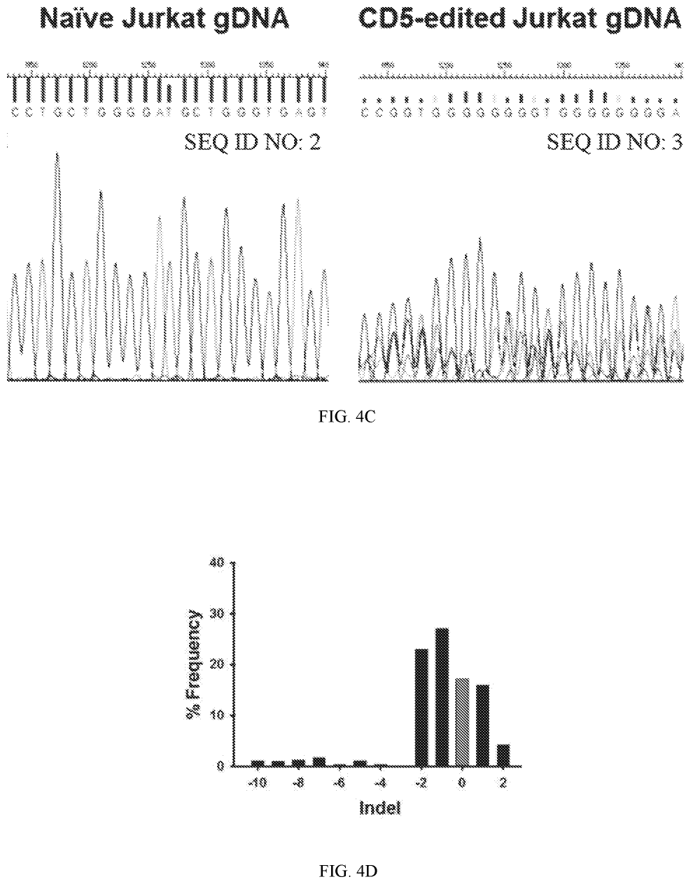

Using nucleoporation, Naive Jurkat T cells were transfected with each CRISPR-Cas9 construct and the percentage of CD5-negative cells five days after transfection was determined. CD5-CRISPR gRNA #2 yielded the greatest increase in CD5-negative Jurkat T cells, resulting in 48% CD5-negative cells, compared to the mock transfected cells, which is a clone that is naturally 15% CD5-negative. gRNA #1 and gRNA #3 resulted in 38% and 24% CD5-negative cells, respectively (FIG. 4A). Using COSMID (CRISPR Off-target Sites with Mismatches, Insertions, and Deletions), a public webtool, one is able to identify sites within the human genome that may be targeted by the CRISPR system. Using the same search parameters, potential off-target sites were identified that could result from using gRNAs #1 and #2; gRNA #1 was predicted to have likely off-target sites in three genes, with one site being within the CD5 gene (separate location from the intended target site), and gRNA #2 was predicted to have likely off-target sites only within the CD5 gene. Given the more efficient CD5 knockout and decreased potential for off-target binding, gRNA #2 was used in subsequent experiments.

Flow sorting allowed for the isolation and expansion of the population of CD5-negative Jurkat T cells from the mixed population of cells edited with CD5-CRISPR gRNA #2. Only 2.1% of sorted cells expressed CD5 (FIG. 4B).

### CD5-Edited CAR-Modified T Cells Have Reduced Self-Activation and Increased CD5-CAR Expression

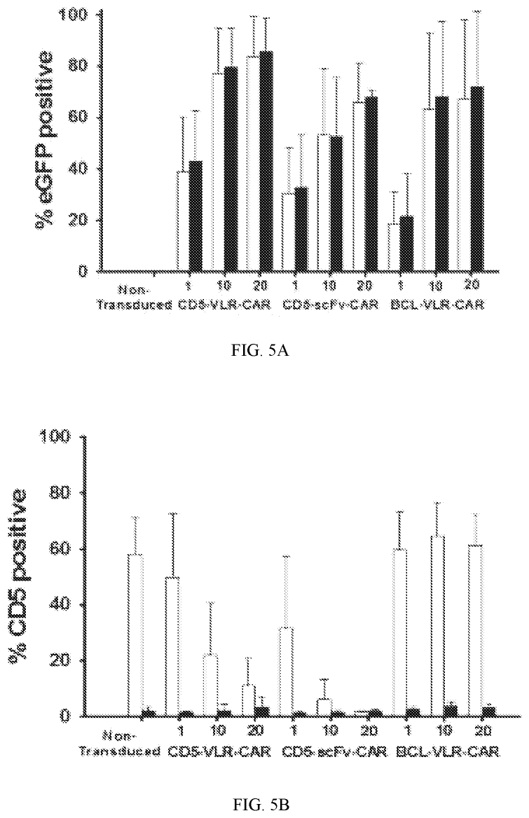

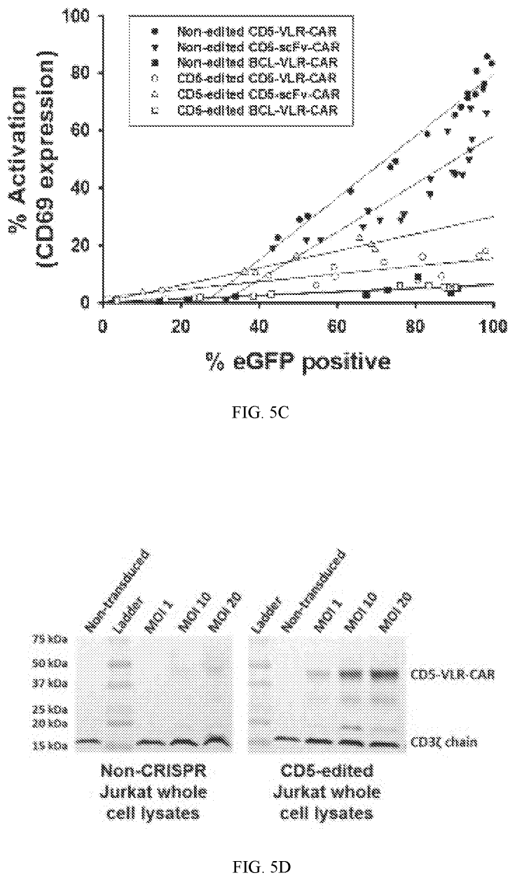

Naive and sorted CD5-CRISPR-edited Jurkat T cells were transduced with the lentiviral vectors encoding eGFP and the CD5-CARs. Additionally, a third lentiviral vector encoding eGFP and BCL-VLR-CAR was used as a negative control. BCL-VLR-CAR expressed in Jurkat T cells does not stimulate T-cell activation in the absence of BCL cells. It may be that both CD5-CARs would activate Naive Jurkat T cells to a greater degree than CD5-edited Jurkat T cells, whereas the BCL-VLR-CAR would stimulate low and equivalent levels of T-cell activation in all Jurkat T cells. eGFP expression was used as a marker of transduced Jurkat T cells to identify the CAR expressing population, and cells were transduced at MOIs of 1, 10 or 20. For all three vectors, there is an increase in eGFP-positive cells as the vector titer increases, and this increase is similar and consistent in both cell populations (FIG. 5A). As the vector amount of CD5-CAR increased, there is a decrease in CD5 expression on non-edited Jurkat T cells (FIG. 5B). This decrease is most pronounced in CD5-scFv-CAR-modified Jurkat T cells. This effect was not observed in BCL-VLR-CAR-modified T cells indicating these results are a consequence of CD5-CAR expression. Furthermore, CD69 expression was compared to eGFP expression in all cell groups. A positive correlation was observed between eGFP expression and activation in both the scFv- and VLR-based CARs, as well as in edited and non-edited cells (FIG. 5C). The increase in activation was dramatically dampened in CD5-edited cells expressing either the CD5-VLR-CAR or the CD5-scFv-CAR. The BCL-VLR-CAR only stimulated very low levels of T-cell activation. Western blot analysis using whole cell lysates collected 9 d after transduction confirmed the decrease in CD5 expression in

CD5-CAR-modified Jurkat T cells compared to Naive Jurkat T cells and BCL-CAR-modified Jurkat T cells. Western blot analysis indicated CD5-edited Jurkat T cells have lower CD5 expression compared to non-edited cells for both transduced and non-transduced cells. If the decrease in CD5 levels is due to interaction between the CD5-CAR and the CD5 cell surface protein then CD5-CAR levels may also be influenced by CD5 expression. Therefore, cells with lower CD5 expression levels will have increased CD5-CAR protein expression due to reduced interactions with the CD5 antigen. To test this, flow cytometry was ran using a CD5-Fc fusion protein consisting of the CD5 antigen fused to the Fc portion of an IgG. Jurkat T cells were stained with the CD5-Fc protein and then stained a second time using an anti-IgG Fc antibody conjugated to phycoerythrin (PE). The CD5-scFv-CAR-modified CD5-edited Jurkat T cells bind CD5-Fc to a greater degree than do CD5-scFv-CAR-modified non-edited Jurkat T cells. This data also indicated potential pseudo-transduction at day 4, however, CD5-Fc binding decreases by day 8 and then appears to plateau. Significant differences are observed early after transduction, however they become less significant after CD5-CAR expression decreases in non-edited cells and normalizes. On day 8 post-transduction, 18.6% of non-edited Jurkat T cells were bound to CD5-Fc protein and eGFP, compared to 35.7% of CD5-edited Jurkat T cells. Experiments in Jurkat T cells serve as a basis for using primary T cells. Primary T cells were expanded in media containing IL-2 and IL-7, and using the same CRISPR-Cas9 system used in Jurkat T cells, CD5 expression was knocked out in 38.6% of our primary T cells. Non-edited and CD5-edited primary T cells were transduced with CD5-scFv-CAR lentiviral vector and measured eGFP and CD5-Fc binding by flow cytometry on day 9 post-transduction. Jurkat T cell data showed increased percentage of CD5-edited cells bound to CD5-Fc protein compared to non-edited cells, with 64.4% CD5-Fc-bound CD5-edited cells, compared to 6.1% CD5- Fc-bound non-edited cells.

The difference in CD5-Fc binding to edited compared to non-edited cells could be a result of steric hindrance from CD5 binding the CAR on non-edited cells, blocking CD5-Fc from binding the CAR, as opposed to reduced CAR expression on these cells. To test this, Western blot analysis was performed on Jurkat whole cell lysates using a CD3zeta antibody to detect endogenous CD3zeta (18 kDa) and CD3zeta in the CAR constructs (48, 55, and 47 kDa in the CD5-VLR-CAR, CD5-scFv-CAR and BCL-VLR-CAR constructs, respectively). Using endogenous CD3zeta as a reference, the CD5-edited Jurkat T cells express both CD5-CARs at greater levels compared to the non-edited Jurkat T cells (FIG. 5D). Furthermore, there is not an effect on BCL-CAR expression when comparing transduced cells with or without CD5 editing indicating non-edited Jurkat T cells have down-regulated CD5-CAR expression.

### CD5-Edited Effector Cells are Efficiently Stimulated by Target T Cells, Which Down-Regulate CD5

Culturing CD5-CAR-modified effector cells with Naïve Jurkat T cells may result in i) an increase in non-edited effector CD5 expression because of competition between CD5 expressed on the CAR-modified cells and CD5 expressed on the target cells, ii) target cell down-regulation of CD5 expression and iii) increased activation of CD5-edited effector cells compared to non-edited cells. Non-edited and

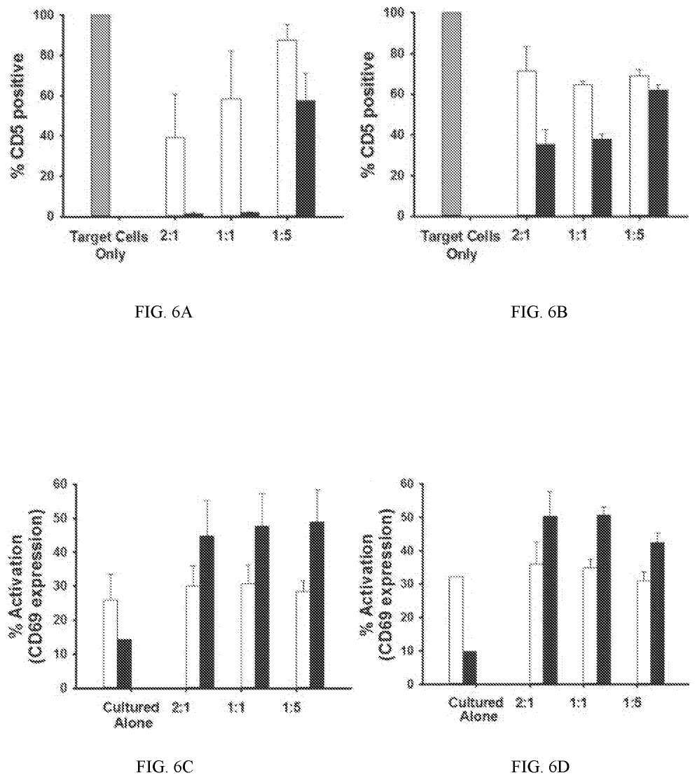

CD5-edited Jurkat T cells were transduced with lentiviral vector encoding CD5-scFv-CAR or CD5-VLR-CAR at an MOI of 5. Flow cytometry five days after transduction confirmed eGFP expression, as well as a decrease in CD5 expression on the non-edited Jurkat T cells. Naïve Jurkat T cells were labeled with Violet Proliferation Dye 450 (VPD450) to distinguish target cells from effector cells, and subsequently cultured with the CAR-modified effector cells at E:T ratios of 2:1, 1:1 and 1:5. After 24 hours, cells were collected and flow cytometry was used to measure CD5 expression on the effector and target cells, as well as CD69 expression on the effector cells. CD5 expression was low in effector cells in edited and non-edited transduced cells when co-cultured with target cells, showing there is little effect on CD5 expression on the effector cells during co-culture. To compare CD5 expression in the target cells, the level of CD5 expression in VPD450-labeled Naïve Jurkat T cells cultured alone was set as the baseline CD5 expression in the target cells. When in culture with CD5-scFv-CAR-(FIG. 6A) and CD5-VLR-CAR modified effector cells (FIG. 6B), CD5 expression decreased in the target cells, with a greater decrease observed in target cells cultured with the CD5-scFv-CAR-modified cells. At E:T ratios of 2:1 and 1:1, there is a significant difference in target cell CD5 expression between the groups cultured with CD5-edited CD5-scFv-CAR-effector cells (FIG. 6A) and CD5-edited CD5-VLR-CAR-effector cells (FIG. 6B). Additionally, significant differences in target cell CD5 expression were found at all E:T ratios comparing the non-edited effector cell group and the CD5-edited effector cell group. However, at low E:T ratios (high percentage of target cells relative to effector cells), the decrease in CD5 expression was less pronounced (FIG. 6A and FIG. 6B, E:T ratio of 1:5 p=0.028 and p=0.045 in CD5-scFv-CAR-effector cell cultures and CD5-VLR-CAR effector cell cultures, respectively). These results show CD5-edited CAR-modified effector T cells have increased association with the target cells compared to non-edited CAR-modified effector T cells, which results in the dramatic decrease in CD5 expression on the target cells. To determine if there are differences in effector cell activation, CD69 expression was measured. At all E:T ratios, CD5-edited CD5-scFv-CAR-(Figure 6C) and CD5-edited CD5-VLR-CAR-modified (FIG. 6D) effector T cells had a significant increase in activation compared to their activation prior to culture with naive target cells (FIG. 6C and 6D). A control experiment measuring the same parameters using non-CAR-modified, CD5-edited effector cells demonstrated the cells alone had no effect. This data illustrates CD5-edited effector T cells have increased interactions with target cells compared to non-edited effector T cells, which results in an increase in effector cell activation.

### CD5-scFv-CAR NK-92 Cells are Superior to CD5-VLR-CAR NK-92 Cells in Delaying Disease Progression in a Xenograft T-Cell Leukemia Mouse Model

To further compare the cytotoxic potential of the two CD5-CAR structures, the efficacy of the CD5-CAR expressing NK-92 cells were tested in a T-cell leukemia xenograft mouse model. Luciferase-expressing Jurkat T cells were used to establish the leukemia model, which allowed for monitoring of tumor burden using bioluminescence imaging. Treatment was started seven days after tumor injection. NK-92 cells were injected twice weekly for a total of 4 doses without IL-2 supplementation. The twice-weekly dosing regimen was based on our experiments showing non-irradiated NK-92 cells, in the absence of IL-2, do not persist in the peripheral blood beyond three days, and show no evidence of engraftment in the bone marrow. A significant decrease in tumor burden was evident in the CD5-scFv-CAR NK-92 treatment group at Day 21. Significance for multiple comparisons tests by Holm-Sidak method was shown for CD5-scFv-CAR vs saline, and CD5-scFv-CAR vs Naive NK-92 groups, but not for the CD5-scFv-CAR vs CD5-VLR-CAR group. A similar overall trend was observed at days 14 and 28 in terms of disease burden; however, the one-way ANOVA test was underpowered to compare all groups. Although only modest effects were observed, due to the cell dose and persistence of the NK-92 cells, the scFv-CAR-treated group had a significant advantage in survival compared to all three other groups with a median survival of 49 d compared to 40, 41, and 42 days for the saline, Naive NK-92 and CD5-VLR-CAR NK-92 groups, respectively. In contrast, the CD5-VLR-CAR-NK-92 mice did not exhibit a significant survival advantage over the saline- and Naive NK-92-treated groups.

### Generation of CAR Encoding Lentiviral Vector.

High titer, recombinant, self-inactivating (SIN) HIV lentiviral vector was produced using a four-plasmid system. The expression plasmid encoding the CD5-CAR constructs and BCL-VLR-CAR construct, as well as packaging plasmids containing the gag, pol, and envelope (VSV-g) genes were transiently transfected into HEK-293 T cells by calcium phosphate transfection. Cells were cultured in DMEM supplemented with 10% FBS and 1% penicillin/streptomycin. Twenty-four hours after transfection, the cell culture medium was replaced with fresh medium. At 48 and 72 hours the vector supernatant was collected, filtered through a 0.22 mm filter and stored at −80° C. After the final collection, the vector supernatant was pooled and concentrated overnight via centrifugation at 10,000×g at 4° C. Pelleted vector was then re-suspended in serum-free StemPro media. Titering was performed on HEK-293 T cell genomic DNA using quantitative polymerase chain reaction (qPCR). Titers of the concentrated recombinant viral vectors were about 1×10⁷ TU/mL.

### Lentiviral Vector Transduction of Cell Lines.

Transduction of recombinant HIV-1-based lentiviral vector particles was carried out by incubating cells with vector in appropriate culture medium supplemented with 6 mg/mL polybrene, unless otherwise stated. Twenty-four hours after transduction, culture medium was replaced with fresh medium. The transduced cells were then cultured for at least 3 d before being used for downstream applications. Jurkat T cells were transduced at multiplicity of index (MOI) ranging from 1 to 20.

### Lentiviral Vector Spinoculation of Primary T Cells.

Transduction of recombinant HIV-1-based lentiviral vectors was carried out by incubating cells with vector in appropriate culture medium supplemented with 5 mg/mL polybrene and then centrifuged at 3000 RPM for 2.5 hours. Twenty-four hours after spinoculation, culture medium was replaced with fresh medium. The transduced cells were then cultured for at least 3 d before being used for downstream applications.

### Transfection of Jurkat T Cells and Primary T Cells.

Jurkat T cells and primary T cells were transfected using the Lonza Nucleofector

2b Device and the Amaxa Cell Line Nucleofector Kit V or the Amaxa Human T Cell Nucleofector kit, respectively, according to the manufacturer's protocol. Cells were transfected with 6 mg of a single plasmid CRISPR Cas9 system encoding both the guide RNA (gRNA) and Cas9. By day 5 post-transfection, the CD5 knockout was confirmed using BD LSRII Flow Cytometer.

### Co-Culture Assay Using CAR-Modified Effector T Cells and Naïve Target T Cells.

Naïve and CD5-edited Jurkat T cells were transduced by incubating with high titer, recombinant, self-inactivating (SIN) lentiviral vectors encoding eGFP-P2A-CD5-scFv-CAR or eGFP-P2A-CD5-VLR-CAR at MOI 5. After 24 hours, culture medium was replaced with fresh medium. On day 5 after transduction, flow cytometry using BD LSRII Flow Cytometer confirmed eGFP expression. The same day, transduced cells were cultured with Naïve Jurkat T cells labeled with Violet Proliferation Dye 450 (VPD450) at effector (E) to target (T) ratios of 2:1, 1:1 and 1:5. The final concentration of each culture was 5×10⁵ cells/mL. Naïve Jurkat T cells were labeled according to the manufacturer's protocol. Flow cytometry was used to analyze changes in CD5 on the effector and target cells, as well as CD69 expression on effector cells at 24 hours after initiation of the co-culture.

Generation of a T-Cell Leukemia Murine Xenograft Model and Treatment with CD5-CAR Expressing NK-92 Cells

NOD/SCID/IL2Rgnull (NSG) mice were purchased from The Jackson Laboratory (Bar Harbor, Me.) and were maintained in a specific pathogen-free environment. Mice were cared for according to the established principles of the Institutional Animal Care and Use Committee (IACUC) and all animal protocols were approved by the IACUC. A luciferase-expressing Jurkat T-cell leukemia cell line was kindly provided to us by Dr. Douglas Graham (Atlanta, Ga.). To determine the treatment dosing regimen with NK-92 cells, NSG mice were injected with non-irradiated CD5-scFv-CAR NK-92 cells without supplementation of IL-2 and followed persistence of the NK-92 cells over time. Mice were evaluated for evidence of NK-92 cells by flow cytometry in peripheral blood, bone marrow and spleen 1, 3, and 18 d post injection. Based on results from this experiment, a twice-weekly dosing regimen for non-irradiated NK-92 cells without IL-2 supplementation was established. Seven- to nine-week-old NSG mice were then intravenously injected with 2×10⁶ luciferase-expressing Jurkat T cells on day 0 to establish disease. Cells were re-suspended in 100 uL phosphate buffered saline (PBS) prior to injection. Treatment was started on day 7 after tumor injection. There were four treatment groups; mice either received PBS (control), unmodified naive NK-92 cells, CD5-VLR-CAR NK-92 cells or CD5-scFv-CAR NK-92 cells. For mice receiving cells, each treatment consisted of 10⁷ NK-92 cells re-suspended in 100 uL PBS administered intravenously via a retro-orbital injection. Each mouse received 4 treatments on days 7, 11, 14 and 18. Mice underwent in vivo bioluminescence imaging every seven days to monitor tumor burden. Animals were monitored frequently and were euthanized upon signs of leukemia progression (weight loss >20%, decreased activity, and/or hind limb paralysis).

Increased VLR-CAR and scFv-CAR Protein Expression in CD-5-Edited T Cwells at All MOIs Compared to That of Non-Edited Jurkat T Cell

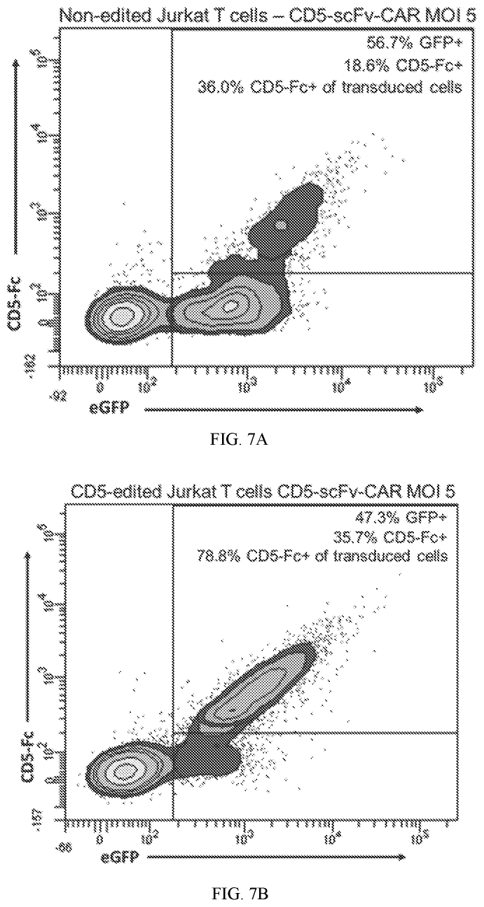

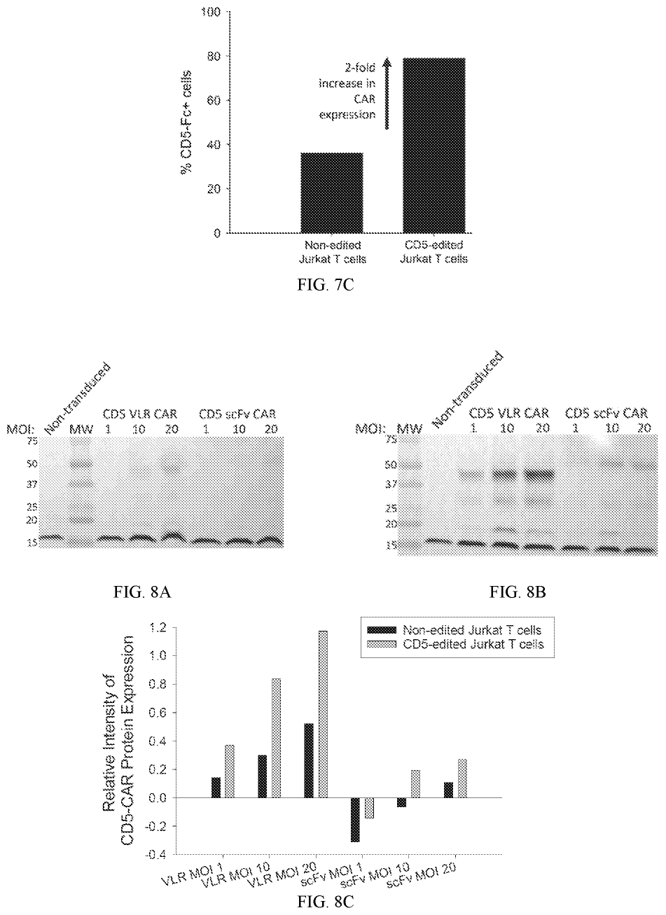

A non-edited and CD5-edited T cell line was transduced with an anti-CD5 scFv CAR at an MOI 5. Flow cytometry was used to measure GFP and CD5-Fc cellular surface expression, which is a measure of CAR expression, five days after transduction. At similar levels of transduction, as measured by GFP expression (FIGS. 7A shows non-edited cells and FIG. 7B shows edited T cells), CD5-edited T cells demonstrate at least a 2-fold increase in CAR expression compared to that of non-edited T cells (FIG. 7C). Additionally, whole cell lysates were isolated from non-edited and CD5-edited T cells that were transduced with CD5 VLR CAR and CD5 scFv CAR lentiviral vectors at MOIs 1, 10 and 20. CAR protein expression was measured by Western blot using anti-CD3zeta antibody, which confirmed greater CAR protein expression in CD5-edited T cells compared to that in non-edited T cells. Endogenous CD3zeta is detected at 18 kDa, and CD3zeta in the CAR constructs are 48 and 55 kDa in the CD5 VLR CAR and CD5 scFv CAR, respectively (FIGS. 8A and 8B). Quantification of the intensity of bands relative to endogenous CD3zeta shows increased VLR-CAR and scFv-CAR protein expression in CD5-edited T cells at all MOIs compared to that of non-edited Jurkat T cell (FIG. 8C).

1. A method of treating cancer comprising:
isolating T-cells from a subject;
modifying the isolated T-cells such that expression of a T-cell antigen is reduced;
inserting a vector into the T-cells, wherein the vector encodes and expresses a chimeric antigen receptor comprising a T-cell antigen recognition domain under conditions such that the T-cells express the antigen recognition domain providing transduced T-cells, wherein reduced expression of the T cell antigen results in an increased expression of a chimeric antigen receptor comprising the T cell antigen recognition domain on the T cells compared to T cells wherein the expression of the T cell antigen is not altered or reduced; and
administering an effective amount of transduced T-cells to the subject, optionally in combination with IL-2, to the subject.

2. The method of claim 1, wherein the T-cell antigen is CD5, CD7, or CD3.

3. A method of treating cancer comprising:
isolating T-cells from a subject;
modifying the isolated T-cells such that expression of CD5 is reduced;
inserting a vector into the T-cells, wherein the vector encodes and expresses a chimeric antigen receptor comprising a CD5 antigen recognition domain under conditions such that the T-cells express the a CD5 antigen recognition domain providing transduced T-cells; and
administering an effective amount of transduced T-cells to the subject, optionally in combination with IL-2, to the subject.

4. The method of claim 3, wherein reduced expression of CD5 results in an increased expression of a chimeric antigen receptor comprising a CD5 antigen recognition domain on the T cells compared to T cells wherein the expression of CD5 is not altered or reduced.

5. The method of claim 3, wherein modifying the isolated T-cells such that expression of CD5 is reduced comprises inserting a vector into the T-cells, wherein the vector encodes and expresses a Cas nuclease and a guide RNA that targets a sequence for cleaving, nicking, or blocking expression of the CD5 gene or CD5 mRNA.

6. The method of claim 5, wherein the guide RNA comprises AGCGGTTGCAGAGACCCCAT (SEQ ID NO: 5).

7. The method of claim 3, wherein modifying the isolated T-cells such that expression of CD5 is reduced comprises inserting into the T-cells mRNA that encodes a Cas nuclease and a guide RNA that targets a sequence for cleavage of nicking in the CD5 gene or CD5 mRNA.

8. The method of claim 7, wherein the guide RNA comprises AGCGGTTGCAGAGACCCCAT (SEQ ID NO: 5).

9. The method of claim 3, wherein modifying the isolated T-cells such that expression of CD5 is reduced comprises inserting a vector or mRNA into the T-cells, wherein the vector or mRNA encodes and expresses a short hairpin RNA capable of reducing CD5 mRNA expression.

10. The method of claim 3, wherein modifying the isolated T-cells such that expression of CD5 is reduced comprises inserting double stranded RNA oligonucleotides into the T-cells wherein the RNA is capable of reducing CD5 mRNA expression by RNA interference (RNAi).

11. The method of claim 3 wherein the T-cells are obtained from autologous peripheral blood lymphocytes (PBL) of the subject.

12. The method of claim 3, wherein administering an effective amount of transduced T-cells to the subject is after administering a lymphodepleting regimen to the subject.

13. The method of claim 12, wherein the lymphodepleting regimen is non-myeloablative.

14. The method of claim 12, wherein the lymphodepleting regimen comprises administering cyclophosphamide, fludarabine, or a combination thereof.

15. The method of claim 3, wherein the CD5 antigen recognition domain comprises EIQLVQSGGGLVKPGGSVRISCAASGYTFTNYGMNWVRQAPGKGLEWMGWINTHTGE PTYADSFKGRFTFSLDDSKNTAYLQINSLRAEDTAVYFCTRRGYDWYFDVWGQGTTVT VSSGGGGSGGGGSGGGGSDIQMTQSPSSLSASVGDRVTITCRASQDINSYLSWFQQKPG KAPKTLIYRANRLESGVPSRFSGSGSGTDYTLTISSLQYEDFGIYYCQQYDESPWTFGGG TKLEIK (SEQ ID NO: 8).

16. A polypeptide comprising EIQLVQSGGGLVKPGGSVRISCAASGYTFTNYGMNWVRQAPGKGLEWMGWINTHTGE PTYADSFKGRFTFSLDDSKNTAYLQINSLRAEDTAVYFCTRRGYDWYFDVWGQGTTVT VSSGGGGSGGGGSGGGGSDIQMTQSPSSLSASVGDRVTITCRASQDINSYLSWFQQKPG KAPKTLIYRANRLESGVPSRFSGSGSGTDYTLTISSLQYEDFGIYYCQQYDESPWTFGGG TKLEIK (SEQ ID NO: 8).

17. A nucleic acid encoding a polypeptide of claim 16.

18. A vector comprising a nucleic acid of claim 17 in operable combination with a promoter.

19. A fusion protein comprising a polypeptide of claim 16.

20. The fusion protein of claim 19 comprising a transmembrane domain, at least one co-stimulatory domain, and a signaling domain.


SEQUENCE LISTING (10 sequences)

SEQ ID NO: 1 - protein, 348 aa, Homo sapiens
RLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNP

SEQ ID NO: 2 - DNA, 23 bp, Artificial
  Synthetic construct
cctgctggggatgctgggtgagt

SEQ ID NO: 3 - DNA, 21 bp, Artificial
  Synthetic construct
ccggtggggggtgggggggga

SEQ ID NO: 4 - DNA, 3 bp, Artificial
  Synthetic construct
  misc_feature (1)..(1): n is a, c, g, or t
ngg

SEQ ID NO: 5 - DNA, 20 bp, Artificial
  Synthetic construct
agcggttgcagagaccccat

SEQ ID NO: 6 - DNA, 1298 bp, Artificial
  Synthetic construct
atgtacaggatgcaactccgtcttgcattgcactaagtcttgcacttgtcacgaattcgggcgcgccttgtccttcacagtgctcctgcagcggaaccgaggtccattgtcagagaaaatccctggcttcagtccctgccggaatcccaaccacaacaagggtgctgtacctgcacgtcaacgagattactaagttcgaaccaggagtgtttgaccgcctggtcaacctgcagcagctgtatctgggaggaaatcagctgagcgccctgccagacggcgtgttcgatcgactgactcagctgaccagactggatctgtacaacaatcagctgaccgtgctgcctgccggggtctttgaccgactggtgaatctgcagacactggatctgcacaacaatcagctgaagtctatccccagaggcgcattcgacaacctgaaaagtctgacccatatttggctgtttgggaatccttgggactgcgcctgtagcgatatcctgtatctgtccggatggctgggacagcatgcagggaaagagcagggacaggctgtctgctctggcaccaacacacccgtgcgggctgtcaccgaggcatcaacatccccatcaaagtgtcctggctacgtggcaacaaccagatctgctagcgagcagaagctgatcagcgaggaggacctggacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactcccaggaggcctgggccaacccgcaagcattaccagccctatgccccaccacgcgacttcgcagcctatcgctccagcaggagcgcagacgctcccgcgtaccagcagggccagaaccagctctataacgagctcaatctaggacgaagagaggagtacgatgttttggacaagagacgtggccgggaccctgagatgggaggcaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctga

SEQ ID NO: 7 - DNA, 1439 bp, Artificial
  Synthetic construct
atgtacaggatgcaactcctgtcttgcattgcactaagtcttgcacttgtcacgaattcgggcgcgcctgaaattcagttggtgcaaagcggaggtggccttgtgaagccaggaggcagtgtgcgaattagttgtgcagcctccggttacacgttcaccaactatggcatgaactgggtgagacaggcccccggcaaggggttggaatggatgggctggattaacacacatacgggcgaaccgacatacgccgacagctttaaaggtcgatttacttttagcttggacgattccaaaaatacggcatacctgcaaataaactcactgcgggcagaggatacggccgtatatttttgtacgcggagagggtacgattggtactttgatgtctggggacaggggacgacagtaaccgtgtctagtggcgggggaggatcaggtggtggcggtagcggtggaggtggaagtgatatccagatgacacaatcaccgagttccctgtccgcgtcagtaggggatcgggtgacaattacatgtagagcatctcaagacatcaatagctacctgagctggtttcagcaaaagcccggaaaagctccgaaaactctgatttatcgggccaatcgccttgagtctggggtgccaagtagattttcaggctccgggagcgggacggactatacgttgaccatatcaagtcttcagtacgaggacttcgggatatactattgccaacagtacgatgagagcccgtggaccttcgggggtgggacaaagttggagatcaaagctagcgagcagaagctgatcagcgaggaggacctggacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactcccaggaggcctgggccaacccgcaagcattaccagccctatgccccaccacgcgacttcgcagcctatcgctcagcaggagcgcagacgctcccgcgtaccagcagggccagaaccagctctataacgagctcaatctaggacgaagagaggagtacgatgttttggacaagagacgtggccgggaccctgagatgggaggcaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgc

SEQ ID NO: 8 - protein, 240 aa, Artificial
  Synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 9 - DNA, 401 bp, Artificial
  Synthetic construct
gactcttcgcgatgtacgggccagatatacgcgtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagcggttgcagagaccccatgttttagagctagaaatagcaagttaaaataaggctagtccgttatcaacttgaaaaagtggcaccgagtcggtgctttttt

SEQ ID NO: 10 - DNA, 80 bp, Artificial
  Synthetic construct
accatgcccatggggtctctgcaaccgctggccaccttgtacctgctggggatgctggtcgcttcctgcctcggacggct